(19) United States
(12) Patent Application Publication
(10) Pub. No.: US 2026/0053767 A1
(43) Pub. Date: Feb. 26, 2026

(54) Hepatoprotective Compositions And Methods

(71) Applicant: Jemyll, Ltd., London (GB)

(72) Inventors: Giulia Angelini, Rome (IT); Matthew Titlow, Carlsbad, CA (US); Timothy Shawn Avila, San Juan Capistrano, CA (US)

(73) Assignee: Jemyll, Ltd., London (GB)

(21) Appl. No.: 18/864,744

(22) PCT Filed: May 8, 2023
(86) PCT No.: PCT/US2023/066740

§ 371 Date: Nov. 11, 2024

Related U.S. Application Data
(60) Provisional application No. 63/341,913, filed on May 13, 2022.
(60) Provisional application No. 63/359,587, filed on Jul. 8, 2022.

(51) Int. Cl. A61K 31/20; A61K 45/06; A61P 1/16; A61P 3/10

(52) U.S. Cl. CPC A61K 31/20; A61K 45/06; A61P 1/16; A61P 3/10

(57) ABSTRACT

Orally administered low-dose formulations for medium chain di-carboxylic acids have unexpectedly a numerous of beneficial cytoprotective effects in a variety of cells. In particular, contemplated formulations compositions and methods reduce, reverse, and/or prevent liver inflammation, hepatic steatosis, and/or liver fibrosis in NASH, improve blood glucose control, and provide additional cytoprotective benefits such as stimulation of mitochondrial biogenesis, increased SIRT and NAMPT levels, increased NAD+, antioxidant capacity, and/or reduction of DNA damage.

### PRIORITY CLAIM

This application claims priority to our co-pending U.S. provisional patent application with Ser. No. 63/341,913, which was filed on May 13, 2022, and U.S. provisional patent application with Ser. No. 63/359,587, which was filed on Jul. 8, 2022. Each of these applications are incorporated by reference herein in its entirety.

### SEQUENCE LISTING

The content of the XML text file of the sequence listing named 104026.0081PCT.xml, which is 11,610 bytes in size was created on Apr. 28, 2023 and electronically submitted via EFS-Web along with the present application, and is incorporated by reference in its entirety.

### FIELD OF THE INVENTION

The field of the invention is compositions and methods for treatment and prevention of sequelae of lipid accumulation in the liver, especially as it relates to low-dose medium chain dicarboxylic acid mediated effects.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Beside their well-known roles in antiseptics, coatings, painting materials, corrosion inhibitors, surfactants, and engineering plastics, dodecanoic diacid (also known as DDDA or DDA) has also been reported as a readily convertible source of various energy substates such as succinyl-CoA and acetyl-CoA and ATP where DDDA is orally administered in large quantities. Notably, relatively large oral doses of DDDA (>20 g) also had a hypoglycemic effect in individuals diagnosed with non-insulin-dependent diabetes mellitus (NIDDM), but no corresponding effects in healthy individuals (see U.S. Pat. Pub. No. 2011/0002900). Likewise, where DDDA was administered to individuals diagnosed with NIDDM by intravenous infusion, plasma glucose levels significantly decreased in NIDDM patients during infusion (see e.g., Nutrition 1998 April; 14(4): 351-7).

Notably, in view of the rapid metabolic conversion of the DDDA in the liver, there are no published reports with respect to the physiological effect of DDDA on hepatocytes and liver tissue. However, it has been reported that the chemically very closely related corresponding monocarboxylic acids (i.e., lauric and palmitic acid) had significant adverse effects on the liver and other tissues. Among other things, lauric and palmitic acid were reported to increase adipose tissue inflammation, to induce insulin resistance, and to precipitate non-alcoholic fatty liver disease (see e.g., Biology (Basel). 2020 Oct. 22;9(11):346).

Non-alcoholic fatty liver disease (NAFLD) and non-alcoholic steatohepatitis (NASH) have become a common condition and are associated with liver fibrosis that can progress to cirrhosis and liver cancer. In the US alone, the number of NAFLD cases are expected to grow from 83 million in 2015 to 101 million in 2030, with 27% of cases meeting the criteria for NASH. The rising disease prevalence is accompanied by an increased number of individuals with both cirrhosis and end-stage liver disease, needing liver transplantation. Nonalcoholic steatohepatitis (NASH) is a pro-inflammatory state that leads to the activation of hepatocytes, Kupffer cells (KCs) and hepatic stellate cells (HSCs). Activated HSC undergo a phenotypic switch and deposit an excessive amount of extracellular matrix that alters the normal liver architecture and leads to liver fibrosis.

Unfortunately, there are no currently FDA approved drugs to treat or reverse these conditions, and numerous herbal extracts have been reported as having at least some beneficial effects (see e.g., US20100074975, US20120171312, US20100086627). However, such formulations are often poorly defined and tend to be less effective than desired.

Thus, even though various composition and methods for treatment and prevention of lipid accumulation in the liver and liver injury due to lipid accumulation, all or almost all of them suffer from several disadvantages. Therefore, there remains a need for improved compositions and methods for treatment and prevention of lipid accumulation in the liver and liver injury due to lipid accumulation.

### SUMMARY OF THE INVENTION

The inventive subject matter is directed to various nutritionally and/or pharmaceutically acceptable compositions and methods that use medium chain dicarboxylic acid at low dosages to prevent and even significantly reduce liver fibrosis in NASH. Such finding was particularly unexpected as the therapeutically effective dose was significantly below dosages that were used in NIDDM treatments. In addition to the reduction of fibrosis, it was also unexpectedly discovered that the low doses also had significant ancillary protective effects that provide significant benefits to hepatocytes and other cell types. Moreover, the inventors also discovered that medium chain dicarboxylic acids were also effective in the prevention and/or reduction and/or reversal of the severity of non-alcoholic steatohepatitis (NASH), and even in treatment of NASH. Furthermore, the inventors also contemplate that medium chain dicarboxylic acids may be effective in prevention of Hepatocellular carcinoma (HCC) in healthy liver and livers of NASH/NAFLD individuals.

In one aspect of the inventive subject matter, the inventors contemplate a hepatoprotective composition that includes a nutritionally or pharmaceutically acceptable carrier in combination with a medium-chain dicarboxylic acid, wherein a therapeutically effective unit dose of the medium-chain dicarboxylic acid provides no more than 5% of a standard daily caloric intake of a mammal.

In some embodiments, the composition is formulated for oral administration. For example, contemplated compositions may be formulated as a ready-to-use drink or as a solid supplement or powder. While not limiting to the inventive subject matter, it is generally preferred that the medium-chain dicarboxylic acid is dodecanoic dicarboxylic acid (DDA or DDDA) or decanoic dicarboxylic acid (sebacic acid).

In some examples, the therapeutically effective unit dose provides equal or less than 3%, or less than 2%, or less than 1% of a standard daily caloric intake of a mammal. Viewed from a different perspective, the therapeutically effective unit dose may also amount to equal or less than 5 g, or equal or less than 4 g, or equal or less than 3 g, or equal or less than 2 g, or equal or less than 1 g of the medium-chain dicarboxylic acid. The therapeutically effective unit dose may also amount to between 3-5 g, or between 2-4 g, or between 1-3 g, or between 0.1-2 g, or between 0.01-0.1 g of the medium-chain dicarboxylic acid. Where desired, the composition may further comprise an additional hepatoprotective agent (e.g., silymarin, a milk thistle extract, taurine, guarana, ginseng, tauroursodeoxycholic acid, leucine, erythritol, pyruvate, a pyruvate derivative, selenium, N-acetylcysteine, glutamine, superoxide dismutase, glutathione, theacrine, and/or methylliberine, etc.).

Advantageously, the therapeutically effective dose may reduce risk for or even reverse progression of liver fibrosis in non-alcoholic steatohepatitis (NASH), and/or may reduce a post-prandial glucose spike and/or post-prandial total blood glucose AUC (e.g., in healthy or pre-diabetic individuals). Moreover, the therapeutically effective dose may also stimulate mitochondrial biogenesis, increase SIRT levels, increase NAMPT levels, increase intracellular NAD+ levels, maintain antioxidant capacity, and/or reduce DNA damage.

Therefore, the inventors also contemplate a method of reducing the risk for or the progression of liver inflammation, hepatic steatosis, and/or liver fibrosis in non-alcoholic steatohepatitis (NASH) in an individual. Such methods will typically include a step of administering a therapeutically effective dose of a medium-chain dicarboxylic acid to the individual in need thereof. wherein the therapeutically effective dose provides no more than 5% of a standard daily caloric intake of the individual. The inventors further contemplate a method of reversing liver inflammation, hepatic steatosis, and/or liver fibrosis in non-alcoholic steatohepatitis (NASH) in an individual, wherein the method comprises administering a therapeutically effective dose of a medium-chain dicarboxylic acid to the individual in need thereof; wherein the therapeutically effective dose provides no more than 5% of a standard daily caloric intake of the individual.

Preferably, but not necessarily, the medium-chain dicarboxylic acid is dodecanoic dicarboxylic acid, and/or the individual is a human. It is further generally preferred that the therapeutically effective dose is orally administered Most typically, the therapeutically effective dose provides equal or less than 3%, or equal or less than 1% of a standard daily caloric intake of a mammal. Thus, the therapeutically effective unit dose may also amount to equal or less than 5 g, or equal or less than 4 g, or equal or less than 3 g, or equal or less than 2 g, or equal or less than 1 g of the medium-chain dicarboxylic acid. The therapeutically effective unit dose may also amount to between 3-5 g, or between 2-4 g, or between 1-3 g, or between 0-2 g, or between 0.01-0.1 g of the medium-chain dicarboxylic acid. As noted above, contemplated compositions may further comprise an additional hepatoprotective agent such as silymarin, a milk thistle extract, taurine, guarana, ginseng, tauroursodeoxycholic acid, leucine, erythritol, pyruvate, a pyruvate derivative, selenium, N-acetylcysteine, glutamine, superoxide dismutase, glutathione, theacrine, and/or methylliberine.

Beneficially, administration of the therapeutically effective dose may also reduce a post-prandial glucose spike and/or post-prandial total blood glucose AUC (e.g., in healthy or pre-diabetic individuals), and in some embodiments, administration of the therapeutically effective dose may stimulate mitochondrial biogenesis, increase SIRT levels, increase NAMPT levels, increase intracellular NAD+ levels, maintain antioxidant capacity, and/or reduce DNA damage.

In still further aspects, the inventors contemplate a method of modulating blood glucose without affecting body weight that includes a step of administering a therapeutically effective dose of a medium-chain dicarboxylic acid to an individual in need thereof, wherein the therapeutically effective dose provides no more than 5% of a standard daily caloric intake of a mammal, and wherein the modulation of blood glucose is a reduction of a post-prandial glucose spike and/or post-prandial total blood glucose AUC (e.g., in healthy or pre-diabetic individuals).

Most typically, the medium-chain dicarboxylic acid is formulated for oral administration, for example, as a ready-to-use drink or as a solid supplement or powder. Preferably, but not necessarily, the medium-chain dicarboxylic acid is dodecanoic dicarboxylic acid. In further embodiments, the therapeutically effective dose provides equal or less than 3% of a standard daily caloric intake of a mammal, and/or the therapeutically effective dose comprises equal or less than 3 g of the medium-chain dicarboxylic acid.

In contemplated methods, administration may also stimulate mitochondrial biogenesis, increase SIRT levels, increase NAMPT levels, increase intracellular NAD+ levels, maintain antioxidant capacity, and/or reduce DNA damage. Advantageously, administration may also reduce risk for or progression of liver fibrosis in non-alcoholic steatohepatitis (NASH).

In yet further aspects, the inventors also contemplate a method of increasing resiliency and/or longevity of a cell that includes a step of exposing the cell to a medium-chain dicarboxylic acid for a time (e.g., for at least 6 hours) and in an amount effective to stimulate mitochondrial biogenesis, increase SIRT levels, increase NAMPT levels, increases intracellular NAD+, maintains antioxidant capacity, and/or reduces DNA damage. Most typically, the medium-chain dicarboxylic acid is dodecanoic dicarboxylic acid, and/or the cell is exposed in vivo after oral administration of the medium-chain dicarboxylic acid.

In further aspects of the inventive subject matter, the inventors contemplate a method of preventing or reducing severity of non-alcoholic steatohepatitis (NASH) in an individual in which a therapeutically effective dose of a medium-chain dicarboxylic acid is administered to the individual, wherein the therapeutically effective dose prevents or reduces severity of non-alcoholic steatohepatitis (NASH) in the individual. Similarly, the inventors also contemplate a method of treating non-alcoholic steatohepatitis (NASH) in an individual in which a therapeutically effective dose of a medium-chain dicarboxylic acid is administered to the individual, wherein the therapeutically effective dose reduces lipid deposits in a liver of the individual diagnosed or suspected to have non-alcoholic steatohepatitis (NASH).

Most typically, the medium-chain dicarboxylic acid is dodecanoic dicarboxylic acid or a pharmaceutically or nutraceutically acceptable salt thereof, and/or the individual is a human. It is furthermore contemplated that the therapeutically effective dose is orally administered. While not limiting to the inventive subject matter, it is generally preferred that the therapeutically effective dose provides equal or less than 5% or equal or less than 3%, or equal or less than 1% of a standard daily caloric intake of the individual. Viewed from a different perspective, the therapeutically effective unit dose may also amount to equal or less than 5 g, or equal or less than 4 g, or equal or less than 3 g, or equal or less than 2 g, or equal or less than 1 g of the medium-chain dicarboxylic acid. The therapeutically effective unit dose may also amount to between 3-5 g, or between 2-4 g, or between 1-3 g, or between 0-2 g, or between 0.01-0.1 g of the medium-chain dicarboxylic acid.

Therefore, the inventors also contemplate a hepatoprotective composition that includes a nutritionally or pharmaceutically acceptable carrier in combination with a medium-chain dicarboxylic acid, wherein the composition is formulated for oral administration at a dosage that is therapeutically effective to prevent or treat non-alcoholic steatohepatitis (NASH) in an individual ingesting the composition. Where desired, the composition may also include at least one additional hepatoprotective agent (e.g., silymarin, a milk thistle extract, taurine, guarana, ginseng, tauroursodeoxycholic acid, leucine, erythritol, pyruvate, a pyruvate derivative, selenium, N-acetylcysteine, glutamine, superoxide dismutase, glutathione, theacrine, and/or methylliberine).

Advantageously, the therapeutically effective dose may further reduce the risk for or the progression of liver inflammation and/or liver fibrosis, and/or may further reduce insulin resistance in the individual.

In further aspects of the inventive subject matter, the inventors contemplate a method of preventing hepatocellular carcinoma, the method comprising administering a therapeutically effective dose of a medium-chain dicarboxylic acid to the individual in need thereof. In preferred embodiments, the individual is a patient having non-alcoholic steatohepatitis (NASH) or non-alcoholic fatty liver disease (NAFLD). Most typically, the medium-chain dicarboxylic acid is dodecanoic dicarboxylic acid or a pharmaceutically or nutraceutically acceptable salt thereof. It is furthermore contemplated that the therapeutically effective dose is orally administered. It is generally preferred that the therapeutically effective dose provides equal or less than 5%, or equal or less than 4%, or equal or less than 3%, or equal or less than 2%, or equal or less than 1% of a standard daily caloric intake of the individual. Viewed from a different perspective, the therapeutically effective unit dose may also amount to equal or less than 5 g, or equal or less than 4 g, or equal or less than 3 g, or equal or less than 2 g, or equal or less than 1 g of the medium-chain dicarboxylic acid. The therapeutically effective unit dose may also amount to between 3-5 g, or between 2-4 g, or between 1-3 g, or between 0-2 g, or between 0.01-0.1 g of the medium-chain dicarboxylic acid.

Various objects, features, aspects, and advantages of the inventive subject matter will become more apparent from the following detailed description of preferred embodiments, along with the accompanying drawings.

### BRIEF DESCRIPTION OF THE DRAWING

FIG. 1 schematically illustrates an exemplary study protocol using Wistar rats on a high fat/high cholesterol diet with or without dodecanedioic acid at low concentration (1% of caloric intake).

FIG. 2 shows exemplary blood glucose challenge results and body weight results for the animals of FIG. 1 with or without dodecanedioic acid at low concentration in their diet.

FIG. 3 shows exemplary results for liver histology for the animals of FIG. 1 after diet with or without dodecanedioic acid at low concentration.

FIG. 4 shows exemplary in vitro results for lipid accumulation in hepatocytes exposed to oleic acid with or without dodecanedioic acid at low concentration.

FIG. 5 shows exemplary data for antioxidant effect of DDA (dodecanedioic acid) in liver cells.

FIG. 6 shows exemplary data for global SIRT activity in DDA exposed liver cells.

FIG. 7 shows exemplary data for PGC-1α levels in DDA exposed liver cells.

FIG. 8 shows exemplary data for NAMPT activity in DDA exposed liver cells.

FIG. 9 shows exemplary data for SIRT3 activity in DDA exposed liver cells.

FIG. 10 shows exemplary data for SIRT1 activity in DDA exposed liver cells.

FIG. 11 shows exemplary data for DNA damage in DDA exposed liver cells.

FIG. 12 shows exemplary data for antioxidant effect of DDA (dodecanedioic acid) in endothelial cells.

FIG. 13 shows exemplary data for global SIRT activity in DDA exposed endothelial cells.

FIG. 14 shows exemplary data for PGC-1α levels in DDA exposed liver cells.

FIG. 15 shows exemplary data for NAMPT activity in DDA exposed endothelial cells.

FIG. 16 shows exemplary data for SIRT3 activity in DDA exposed endothelial cells. FIG. 17 shows exemplary data for DNA damage in DDA exposed endothelial cells.

FIG. 18 schematically depicts the experimental design of the study. In Study 1 (A) DODA was administered together with a high fat high cholesterol diet for 4 weeks. while in Study 2 (B) rats were first fed high fat high cholesterol diet for 9 weeks to develop NASH and then DODA was administered for 4 weeks to assess its reversal.

FIG. 19 depicts exemplary results demonstrating that DODA administration prevents diet-induced insulin resistance and NASH. Panels A, B are time courses and AUC of blood glucose and plasma insulin concentrations during an oral glucose tolerance test. Panel C shows hepatic insulin resistance assessed by HOMA-IR. Data are mean±SEM of data from 10 rats per group. ***P<0.0001; ***P<0.004; ***P<0.015.

FIG. 20 depicts exemplary results demonstrating that DODA administration prevents diet-induced NASH. Panels A-F are representative for Hematoxylin and Eosin (A,B), Oil Red O for lipid staining (C,D) and Picro Sirius Red for fibrosis (E,F) staining of liver sections. Magnification 20× Scale bar: 0.10 mm.

FIG. 21 depicts exemplary results demonstrating that DODA administration reverses diet-induced insulin resistance. Panels A, B: Time courses and AUC of blood glucose and plasma insulin concentrations during an oral glucose tolerance test. Panel C: Hepatic insulin resistance and insulin sensitivity assessed by HOMA-IR. Data are mean±SEM of data from 10 rats per group. ***P<0.004; ***P<0.04.

FIG. 22 depicts exemplary results demonstrating that DODA administration reverses diet-induced insulin NASH. Panel A-F: Representative Hematoxylin and Eosin (A,B), Oil Red O for lipid staining (C,D) and Picro Sirius Red for fibrosis (E,F) staining of liver sections. Magnification 20×. Scale bar: 0.10 mm.

FIG. 23 shows in vitro lipid droplets accumulation in primary hepatocytes by Nile red staining of palmitate treated (0.4 mM) primary hepatocytes incubated with or without DODA 1% (w/vol). Magnification 60×. Scale bar: 50 μm.

FIG. 24 depicts exemplary results demonstrating that DODA induces HSCs apoptosis contributing to the resolution of liver fibrosis. Panels A, B: Flow cytometry analysis of myofibroblastic differentiation of hepatic stellate cells exposed for 3 days to CM from palmitate-treated hepatocytes with (B) or without (A) DODA 1% (w/vol) for 24 hours. Panels C, D: Flow cytometry based analysis of HSCs apoptosis after incubation with CM from palmitate-treated hepatocytes with (D) or without (C) DODA 1% (w/vol), using propidium iodide staining.

FIG. 25 depicts exemplary study design for in vivo study 1 (Prevention, panel A) and in vivo study 2 (Reversion, panel B) showing prevention or reversion of NAFLD using the compositions disclosed herein.

FIG. 26 shows C12 administration prevents diet-induced insulin resistance and NASH. Panel A & B shows time courses and AUC of blood glucose and plasma insulin concentrations during an oral glucose tolerance test. Panel C shows hepatic insulin resistance assessed by HOMA-IR.

FIG. 27 shows that C12 administration prevents diet-induced NASH. Panels A-F: representative Hematoxylin and Eosin (A,B), Oil Red O (C,D) and Picro Sirius Red (E,F) staining of liver sections. Magnification 20×. Scale bar: 0.10 mm.

FIG. 28 depicts exemplary results showing that C12 administration reverses diet-induced insulin resistance. Panel A, B: Time courses and AUC of blood glucose and plasma insulin concentrations during an oral glucose tolerance test. Panel C: Hepatic insulin resistance assessed by HOMA-IR. ***P<0.0001; ***P<0.004; ***P<0.015.

FIG. 29 shows that C12 administration reverses diet-induced insulin NASH. Panles A-F” Representative Hematoxylin and Eosin (A,B), Oil Red O (C,D) and Picro Sirius Red (E,F) staining of liver sections. Magnification 20×. Scale bar: 0.10 mm.

FIG. 30 shows that C12 induces HSCs apoptosis contributing to the resolution of liver fibrosis. Panel A,B: Flow cytometry analysis of myofibroblastic differentiation of hepatic stellate cells exposed for 3 days to CM from palmitate-treated hepatocytes with (B) or without (A) C12 1% (w/vol) for 24 hours. Panel C,D: Flow cytometry based analysis of HSCs apoptosis after incubation with CM from palmitate-treated hepatocytes with (D) or without (C) C12 1% (w/vol), using propidium iodide staining. Panel E: qReal time-PCR analysis of genes associated with fibrosis performed in the liver of rats from in vivo study 1 and 2.

FIG. 31 shows that C12 inhibits citrate uptake reducing citrate transporter SLC13A5. Panel A: In vitro citrate uptake performed on human primary hepatocytes treated with a fixed concentration of citrate (150 μM) and with or without C12 (0.1-1.5% v/w). Panel B: Solute carrier family 13 member 5 (SLC13A5) assessed by qReal time-PCR in primary human hepatocytes treated with palmitate (0.4 mM) and with or without C12 1% (v/w).

FIG. 32 shows that C12 reduces lipid droplets accumulation and DNL in primary hepatocytes. Panel A: Nile red staining of primary human hepatocytes treated with palmitic acid (0.4 mM) with or without C12 1% (w/vol). Magnification 60×. Scale bar: 50 μm. Panel B: De novo lipogenesis enzymes (namely Diacylglycerol O-Acyltransferase 1 (DGAT1), Fatty Acid Synthase (FASN) and Stearoyl-CoA desaturase (SCD1)) assessed in primary human hepatocytes treated with palmitic acid (0.4 mM) and with or without C12 1% (v/w). Panel C: Fatty acid Beta-Oxidation key enzyme (namely carnitine palmitoyltransferase 1A (CPT1A)) assessed in primary human hepatocytes treated with palmitic acid (0.4 mM) and with or without C12 1% (v/w).

FIG. 33 shows that C12 effects on DNL, FAO and citrate uptake in vivo. Panel A: De novo lipogenesis enzymes (namely Diacylglycerol O-Acyltransferase 1 (DGAT1), Fatty Acid Synthase (FASN) and Stearoyl-CoA desaturase (SCD1)) assessed by qReal Time-PCR in the liver of rats from in vivo study 1 and 2. Panel B: Fatty acid Beta-Oxidation key enzyme (namely carnitine palmitoyltransferase 1A (CPT1A)) assessed by qReal Time-PCR in the liver of rats from in vivo study 1 and 2. Panel C: Solute carrier family 13 member 5 (SLC13A5) assessed by qReal time-PCR in the liver of rats from in vivo study 1 and 2.

### DETAILED DESCRIPTION

The inventors have unexpectedly discovered that oral administration of low doses of medium chain dicarboxylic acids had a variety of cytoprotective effects, and low doses were especially effective in reducing or even reversing fibrosis of the liver in NASH, and in reducing post-prandial glucose spike and post-prandial total blood glucose AUC in non-diabetic individuals. Moreover, it was unexpectedly observed that the low doses upon oral administration stimulated mitochondrial biogenesis, increased global SIRT and NAMPT levels (and with that increased intracellular NAD+), helped maintain antioxidant capacity, and reduced DNA damage. In addition, the inventors also discovered that medium chain dicarboxylic acids were also effective in preventing and/or reducing and/or reversing the severity of non-alcoholic steatohepatitis (NASH) in an individual and could even be used to treat NASH in an individual by reducing lipid deposits in a liver of the individual diagnosed or suspected to have non-alcoholic steatohepatitis.

Consequently, and based on the data as presented in more detail below, the inventors contemplate that medium-chain dicarboxylic acids can be used in an oral administration form to counteract liver fibrosis, to prevent or reduce the severity of NASH, to treat NASH, to enhance cellular health, and/or assist in maintenance of blood glucose levels (especially in non-diabetic individuals, e.g., by reducing insulin resistance). Moreover, and especially over extended periods of administration, contemplated compositions may also be effective to promote weight loss (particularly in a population that is overweight or obese).

For example, in one representative embodiment, a nutritional supplement is formulated as a ready-to-use drink or bulk powder that contains about 3 g of dodecanoic dicarboxylic acid (DDDA) in a single dosage unit. The drink is preferably formulated as a flavored aqueous non-alcoholic solution, while the ready-to-use powder may be formulated with an edible and preferably non-caloric or low-caloric carrier (e.g., soluble prebiotic fiber) that can be admixed with a fluid or other food item. In another example, a pharmaceutical formulation for oral administration is formulated as solid tablet(s) to provide as a daily dosage between 0.05 g and 5 g of dodecanoic dicarboxylic acid.

As will be readily appreciated, various medium-chain dicarboxylic acids other that DDDA can be used in contemplated compositions and especially preferred alternate dicarboxylic acids include sebacic acid, and generally dicarboxylic acids having the general formula of (CH₂)ₙ(CO₂H)₂ in which n is preferably an integer between 6 and 12. Moreover, it should be appreciated that the combinations of various medium-chain dicarboxylic acids having different molecular weights are also deemed appropriate. Still further, it should be appreciated that various modifications to the medium-chain dicarboxylic acids are also deemed suitable, and exemplary modifications include addition of a functional group (e.g., hydroxyl group, halogen, amino group, thiol group, etc.) or replacement of a hydrogen in the medium-chain dicarboxylic acids with a functional group. Likewise, contemplated medium-chain dicarboxylic acids may be modified to form mono-or diesters with various groups to modulate absorption, serum half-life, etc. Additionally, it should also be recognized that the medium-chain dicarboxylic acids contemplated herein include all metabolites of the medium-chain dicarboxylic acids and mixtures thereof.

Furthermore, the inventors also found that administration of DDDA to cancer bearing mice is helpful for liver blastomas and potentially NASH associated carcinomas. Thus, DDDA in low doses (5 g or less, or 4 g or less, or 3 g, or less, or 2 g or less, or 1 g or less) may be used for chemoprevention of HCC in healthy liver or livers of NASH/NAFLD individuals, presumably due to reduction of chronic subacute inflammation.

Throughout the present disclosure, the terms “dodecanoic dicarboxylic acid”, “DDA”, “DDDA”, “C12”, and “DODA” are being used interchangeably, and refers to a dicarboxylic acid with the formula (CH₂)₁₀(COOH)₂.

With respect to suitable carriers, it should be noted that al carriers are suitable so long as they are nutritionally, and/or pharmaceutically acceptable. Therefore, especially preferred carriers will include materials suitable for human and animal consumption that may be solid or liquid. For example, solid carriers will typically include all excipients commonly used in the nutritional and pharmaceutical arts such as fillers, binders, disintegrants, etc. where the composition is formulated as a powder, tablet, capsule, or other orally administrable form. On the other hand, where the composition is formulated as a snack or food item, especially preferred formulations will include snack bars, cookies, gummies, etc. Additionally, solid carriers will also include all baked goods where the medium-chain dicarboxylic acids are used to fortify the baked goods to so blunt a blood glucose and/or insulin spike that would otherwise postprandially be observed. In further examples, liquid carriers will include aqueous formulations and soft drinks that may or may not be carbonated, syrups, fruit juices, and flavored beverages, all of which may be packed into small ready-to-use/single-use containers or containers that store multiple dosage units.

Regardless of the particular formulation, it should be appreciated that contemplated compositions and products will include at least one therapeutically effective unit dose of the medium-chain dicarboxylic acid, which will typically (but not necessarily) provide no more than 5% of the standard daily caloric intake of a mammal. Viewed from a different perspective, the therapeutically effective unit dose will generally be a dose that is effective to produce a physiologically desirable effect as is described in more detail below. Among other desirable effects, administration of therapeutically effective unit doses over a period of at least 1 week, or at least 2 weeks, or at least 4 weeks, and longer, will include prevention and/or reduction of liver inflammation, NAFLD, NASH, hepatic steatosis, and/or liver fibrosis in individuals diagnosed with NAFLD or NASH, prevention and/or reduction of lipid accumulation in hepatocytes of healthy individuals or individuals at risk for development of NAFLD or NASH, treatment of NAFLD or NASH, reduction of post-prandial glucose spikes and/or post-prandial total blood glucose (especially in non-diabetic or pre-diabetic individuals) as determined by AUC measurement. Additional desirable effects include a variety of cytoprotective effects such as increased mitochondrial biogenesis, increased SIRT levels, increased NAMPT levels, increased intracellular NAD+ levels, maintained antioxidant capacity, and/or reduced DNA damage. In still further embodiments, it is contemplated that the compositions presented herein may also be used to address, alleviate, and/or treat conditions and diseases associated with elevated lipid load, including PCOS.

Consequently, and in at least some embodiments it should be appreciated that administration of DDDA (and other dicarboxylic acids) may be an excellent way to not only treat but also to reverse NASH (and therefore NAFLD). Notably, and as shown in more detail below, liver fibrosis can also be resolved with dicarboxylic acids. Of course, it should be noted that dicarboxylic acids other than DDDA may also provide similar benefits as well given their similar properties. Moreover, it was unexpectedly discovered that dicarboxylic acids had significant effects at a low concentration or dosage, and particularly on liver diseases.

Therefore, in certain embodiments contemplated therapeutically effective unit doses will provide equal or less than 10%, or equal or less than 9%, or equal or less than 8%, or equal or less than 7%, or equal or less than 6%, or equal or less than 5%, or equal or less than 4%, or equal or less than 3%, or equal or less than 2%, or equal or less than 1% of a standard daily caloric intake of a mammal. Most typically, the daily caloric intake for human is about 2,000 calories/day for adult women and about 2,500 calories/day for adult men. Where contemplated compositions are used for pet food, a typical caloric intake for cats is about 200 calories/day for a 10 lb cat, and between 200-1.000 calories for a dog having a body weight of 10-70 lbs.

Viewed from a different perspective, contemplated therapeutically effective unit doses will typically be equal or less than 6 g, or equal or less than 5 g, or equal or less than 4 g, or equal or less than 3 g, or equal or less than 2 g, or equal or less than 1 g, or equal or less than 0.5 g, or equal or less than 0.3 g of the medium-chain dicarboxylic acid.

In this context, it should be especially appreciated that the metabolic or physiological role of contemplated medium chain dicarboxylic acids will significantly change as a function of the dose administered, which was neither recognized nor expected by the skilled artisan. Indeed, while relatively high quantities (e.g., 40 g) of orally administered DDDA will predominantly operate as a source of acetyl-CoA, succinyl-CoA, and ATP, lower doses of intravenous DDDA improved glycemic control in individuals with NIDDM, whereas very low doses as presented herein prevented and even reversed liver fibrosis in NASH, lipid accumulation in hepatocytes, and NASH, and also reduced blood glucose excursions and overall serum concentration (as measured by AUC) in non-diabetic individuals. Additionally, low dosages also provided significant cytoprotective effects in a significant manner as is described in more detail below. Therefore, it should be recognized that medium chain dicarboxylic acids have a dose dependent multi-modal effect. While not limiting to the inventive subject matter, the inventors contemplate that the beneficial effects described herein may be attributable to the interaction of the medium chain dicarboxylic acids (and especially DDDA) with NR113 (nuclear receptor subfamily 1 group 1 member 3), which is a known key regulator of xenobiotic and endobiotic metabolism. Additionally, or alternatively, the beneficial effects described herein may also be attributable to the interaction of the medium chain dicarboxylic acids (and especially DDDA) with HDAC9 (histone deacetylase 9), a member of histone deacetylases, which are known to play a regulatory role in dependence of the metabolic state of the cell. Still further, the inventors also note that the DDDA and compositions containing DDDA promoted mitochondrial fatty acid oxidation, which is believed to assist in weight loss and reduction in hepatic and/or adipocytic lipid load.

As will be readily appreciated, contemplated compositions may also include one or more additional functional ingredients that assist in maintenance of cellular health, and particularly health of liver cells. For example, contemplated hepatoprotective agents include silymarin, a milk thistle extract, taurine, guarana, ginseng, tauroursodeoxycholic acid, leucine, erythritol, pyruvate, a pyruvate derivative, capsaicin, a capsaicin derivative, selenium, N-acetylcysteine, glutamine, superoxide dismutase, glutathione, theacrine, and/or methylliberine. Likewise, additional agents may also assist in control of normal blood glucose levels, and exemplary ingredients will include powdered forms and extracts from cinnamon, ginseng, various probiotics, Aloe vera, Gymnema sylvestre, as well as alpha lipoic acid, and berberine, and trivalent chromium complexed with one or more ligands. Still further contemplated additional agents include those that promote and/or maintain a ketogenic state, and especially preferred agents include beta-hydroxy butyric acid, butyric acid, tributyrin, acetoacetate, etc.

Most typically, contemplated compositions are administered over an extended period of time using at least a single therapeutically effective dose per day. For example, the compositions may be administered over at least 3 days, or at least 7 days, or at least 2 weeks, or at least 4 weeks, or at least 2 months, or at least 3 months, and even longer. Preferably, but not necessarily, administration is together with a meal, but may also be used while fasting.

### Examples

The following examples are provided to illustrate selected physiological markers and/or processes that can be beneficially, and in at least some cases synergistically modulated by the compositions presented herein. However, these examples are not intended to be limiting the inventive subject matter, and additional and/or alternative markers and processes are also expressly contemplated herein. Unless noted otherwise, measurement of these markers is known in the art and will follow well-known protocols.

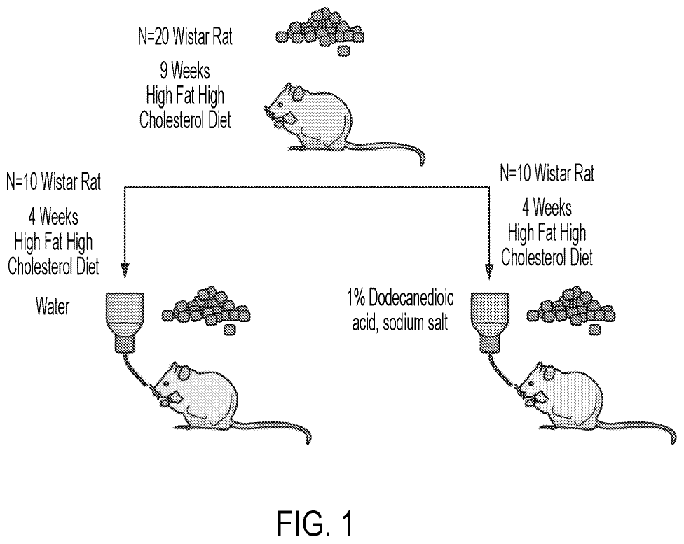

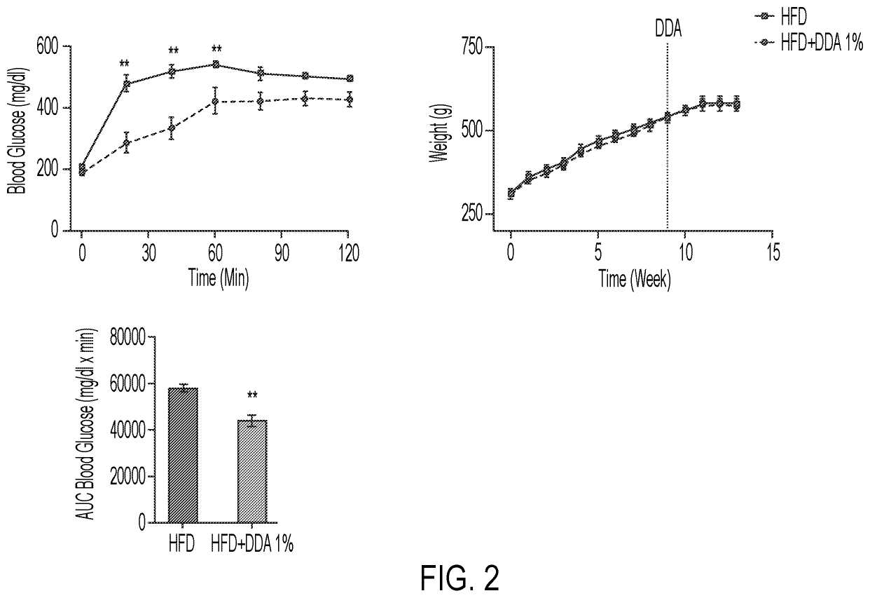

Animal Studies. All in vivo studies used Wistar rats that were kept for 9 weeks on a high fat/high cholesterol diet. The animals were then separated into two groups, a control group that continued the high fat/high cholesterol diet with regular water, whereas the treatment group continued the high fat/high cholesterol diet with water containing DDA (dodecanedioic acid) in an amount of 1% of the caloric intake. A schematic overview of the study is shown in FIG. 1. After all animals were separated in the respective test groups, body weights were recorded, and a glucose challenge test administered. Notably, and as can be seen from FIG. 2, there was no significant difference in the body weight between both groups. However, the response to glucose challenge clearly revealed that glucose metabolism was positively affected by the very low concentration of DDA in the diet. Notably, the blood glucose spike was significantly blunted in the DDA group, and the AUC blood glucose was also significantly reduced as is shown in FIG. 2. Such effect is particularly unexpected as the quantity of DDA in the food was well below any quantity that was reasonably expected.

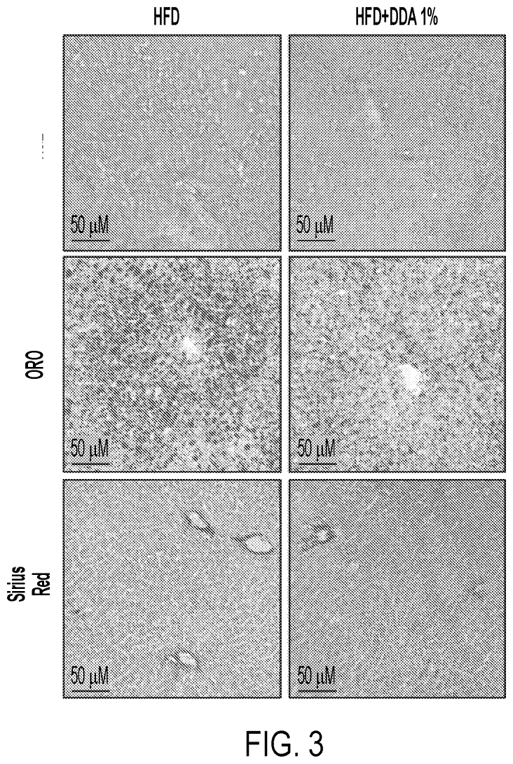

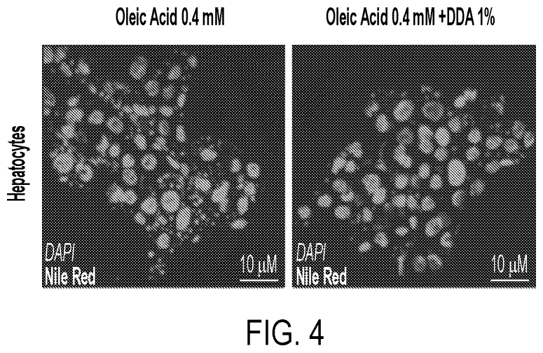

Still further, the inventors also unexpectedly discovered that the low concentration of DDA in the high fat/high cholesterol diet had substantial hepatoprotective effect as is shown in the histopathology analyses of FIG. 3. More specifically, lipid vesicles cell size/distension, and overall tissue morphology was characteristic for non-alcoholic steatohepatitis (NASH) in the liver tissue under H&E stain of the rats fed the high fat/high cholesterol diet, whereas the cells and liver tissue of rats fed a high fat/high cholesterol diet with DDA was significantly closer to normal liver histology. Likewise, when the liver tissues were stained with red oil (ORO) to demonstrate presence of fatty deposits, significant fat deposits were observed in the liver tissue of rats fed a high fat/high cholesterol diet, while the liver tissue of rats fed the same high fat/high cholesterol diet with low quantities of DDA had a normal or near-normal microscopic appearance.

Similarly, where tissue sections were stained with Sirius red to indicate hepatic collagen (which is a marker of fibrosis), it was once more apparent that rats that were fed the high fat/high cholesterol diet had significantly increased fibrosis as compared to those rats where the diet included DDA at low concentrations. While not wishing to be bound by any specific theory or hypothesis, the inventors contemplate that the DDA at the low levels inhibits the trans-differentiation of hepatic stellate cells to myofibroblasts that produce extracellular matrix proteins (including collagen), likely by protecting them from damage due to reactive oxygen species (ROS). Thus, it should be appreciated that the low quantities of DDA in the presence of a fat/high cholesterol diet had substantial hepatoprotective effect despite its apparent lack to contribute to caloric intake in a meaningful manner.

Prior experimental data demonstrated that where DDA contributed significantly to caloric intake (˜15%; equivalent to about 40 g daily DDA intake in human diet), DDA had a modulatory effect on metabolism. Indeed, DDA at these high quantities provided a substantial source for production of acetyl-CoA, succinyl-CoA, and ATP. Therefore, based on these high DDA quantities, any metabolic effect for the quantities noted above (about 1% of caloric intake) were not reasonably expected. Nevertheless, and as was demonstrated in the results of FIG. 2, low quantities of DDA did have a profound effect of glycemic control even in the presence of a high fat/high cholesterol diet.

Based on these unexpected results, the inventors then sought out to determine if DDA had also effects on oxidative stress resilience, protection from DNA damage under oxidative stress, and if DDA could have beneficial effects on epigenetic modification (and especially acetylation via SIRT) and mitochondrial biogenesis. Viewed form a different perspective, DDA was postulated to also affect processes associated with health maintenance during aging and/or during oxidative insult.

To that end, the inventors used endothelial cells (EOMA cells) and liver cells (AML-12 cells) in vitro and quantified various markers in response to exposure of the cells to environmental challenge in the presence and absence of DDA at low concentrations. Unless indicated otherwise, the DDA concentration in the in vitro experiments was 5 mcg per microliter. More specifically, the inventors measured p-H2AX protein levels as a marker for DNA damage, PGC-1α protein levels as a marker for mitochondrial biogenesis, NAMPT enzyme levels as a marker for potential for NAD+ production, and total antioxidant capacity as a marker of cellular health. In addition, the inventors also measured global SIRT activity as a gauge of activity for all SIRT enzymes, and in particular SIRT1 enzyme levels as markers for autophagy and various beneficial processes associated with a caloric restricted diet, and SIRT3 levels as markers for mitochondrial function and longevity.

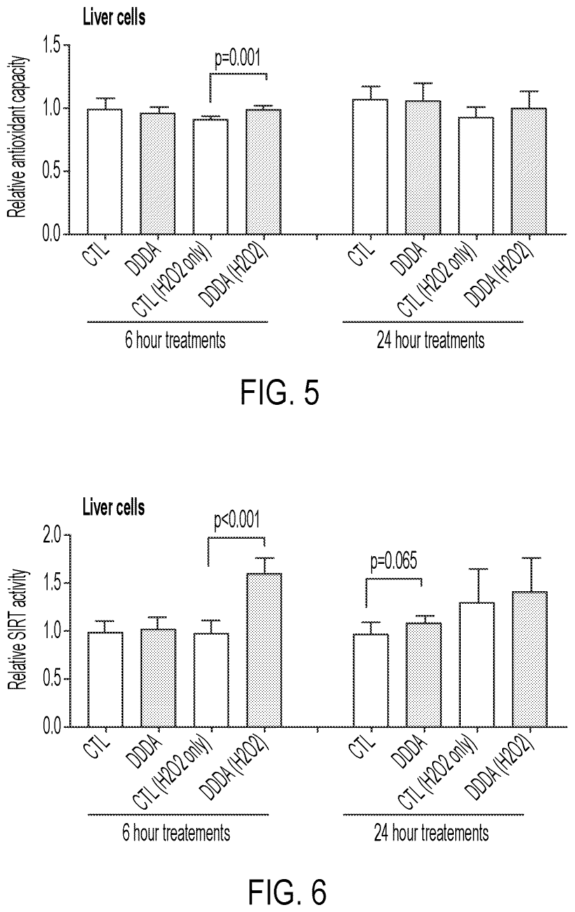

Antioxidant activity in liver cells was tested and exemplary results are shown in FIG. 5. As can be readily seen, the data show that 6 hours of DDDA (in the presence of hydrogen peroxide) maintains cellular total antioxidant capacity (p=0.001). The same trend was evident after 24 hours of treatment in the presence of hydrogen peroxide, albeit this did not reach a level of significance (N=5-6 plates per treatment). Therefore, at least in an acute setting, it was observed that DDA (also referred to as DDDA herein) had potent antioxidant activity after 6 hours, suggesting that DDA provided significant protection and supports cellular health.

In still further experiments, the liver cells were also tested for global SIRT activity and exemplary results are shown in FIG. 6. Here, the data show that 6 hours of DDDA (in the presence of hydrogen peroxide) increased global SIRT activity in a statistically significant manner (p<0.001). The same trend was also evident after 24 hours with no hydrogen peroxide treatments (p=0.065), albeit this did not reach a level of significance (N=5-6 plates per treatment). Thus, and once more in at least an acute setting, DDA significantly increased global SIRT activity, indicating improved stress resilience, autophagy, and prolonged longevity.

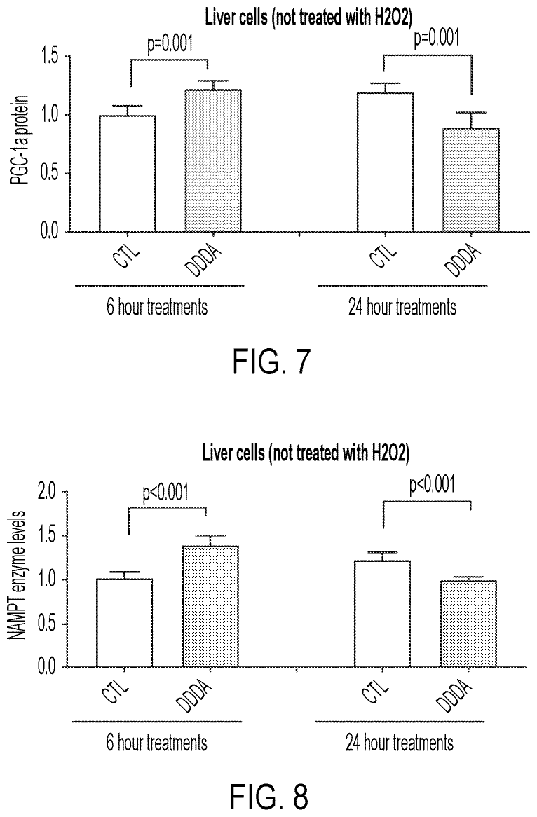

The inventors also evaluated activity of DDA on energy metabolism, and particularly on mitochondrial health and/or mitochondrial biogenesis. The protein followed was PGC-1α, a known marker for mitochondrial biogenesis, and exemplary results are depicted in FIG. 7. Here, the data show that after 6 hours DDDA increased PGC1α levels (p=0.001). Notably, this pattern paradoxically reversed after 24 hours of treatment (N=5-6 plates per treatment). Collectively, these data indicate that the rapid DDDA-induced upregulation in this marker may have led to negative feedback where (by 24-h post-treatment) this metric returns back down to baseline. Such finding is not entirely unexpected as PGC-1α is a transcriptional co-activator and as such tightly regulated. Therefore, the data suggest that DDDA may exert a strong positive stimulus on mitochondrial biogenesis.

To evaluate the effect of DDDA on energy metabolism, and particularly on NAD+ synthesis, the inventors also tested the effect of DDDA on NAMPT in liver cells. FIG. 8 shows exemplary results. As can be seen form the graphs, the data show that after 6 hours of DDDA the NAMPT enzyme levels significantly increased (p=0.001; N=5-6 plates per treatment).

Notably, this pattern paradoxically reversed after 24 hours of treatment. Like the PGC-1α data in FIG. 7 above, the data suggest that the rapid DDDA-induced upregulation in this marker may have led to negative feedback where (by 24-h post-treatment) this metric returns back down to baseline. Therefore, DDDA may have a potential stimulatory effect on NAMPT enzyme regulation, and therefore on NAD+ levels.

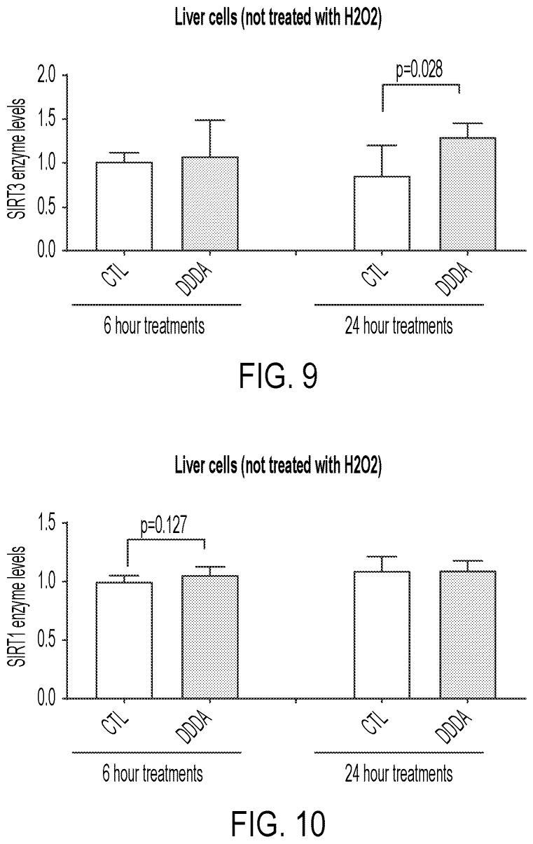

In still further experiments, the inventors also investigated whether or not DDDA could have stimulatory effects on selected SIRT proteins. More specifically, the inventors tested the effects of DDDA on SIRT3 and SIRT1, and exemplary results are shown in FIG. 9 and FIG. 10, respectively. As can be seen from FIG. 9, the data show that after 24 hours of DDDA exposure, SIRT3 protein levels were significantly increased (p=0.028). This may have been why the up-trend was observed in 24-hour DDDA-treated cells with global SIRT activity in FIG. 10 (p=0.065). N=5-6 plates per treatment. On the other hand, and as shown in FIG. 10, the data show that after 6 hours of DDDA exposure, SIRT1 levels trended upwards (p=0.127). Beyond this trend, however, no other observations were evident. N=5-6 plates per treatment.

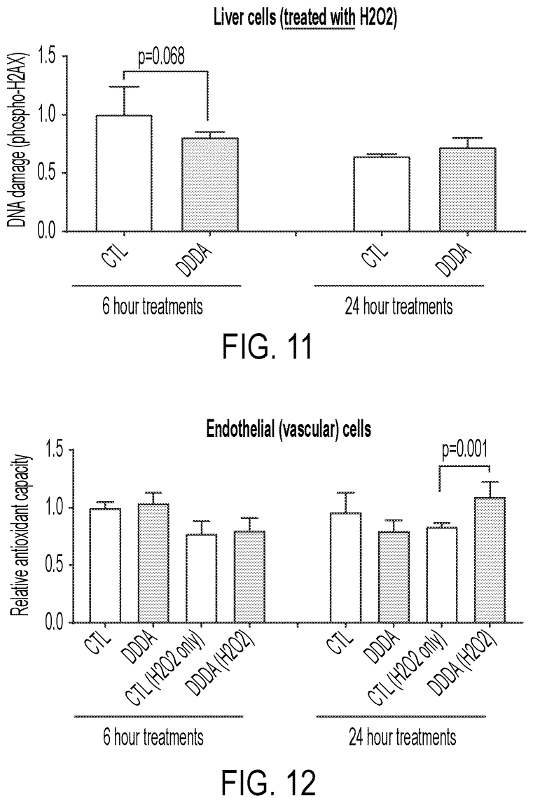

Finally, the inventors also investigated whether or not DDDA could have a protective effect against peroxide induced DNA damage, and exemplary results are shown in FIG. 11. Here, it can be readily seen that that after 6 hours of DDDA exposure a trend towards decreased DNA damage was observed (p=0.068), which is consistent with the data in FIG. 5 for antioxidant effect. N=5-6 plates per treatment.

In yet a further series of experiments, the inventors also sought to confirm whether or not DDDA would have a modulatory effect in endothelial cells using the same markers and experimental conditions as noted above.

With respect to antioxidant capacity, FIG. 12 depicts exemplary results. As can be seen from the data, after 24 hours of DDDA exposure (in the presence of hydrogen peroxide) cellular total antioxidant capacity was maintained (p=0.001). These data confirm and reiterate what was found in liver cells, and warrant examining how DDDA protects against oxidative stress in vivo. N=5-6 plates per treatment.

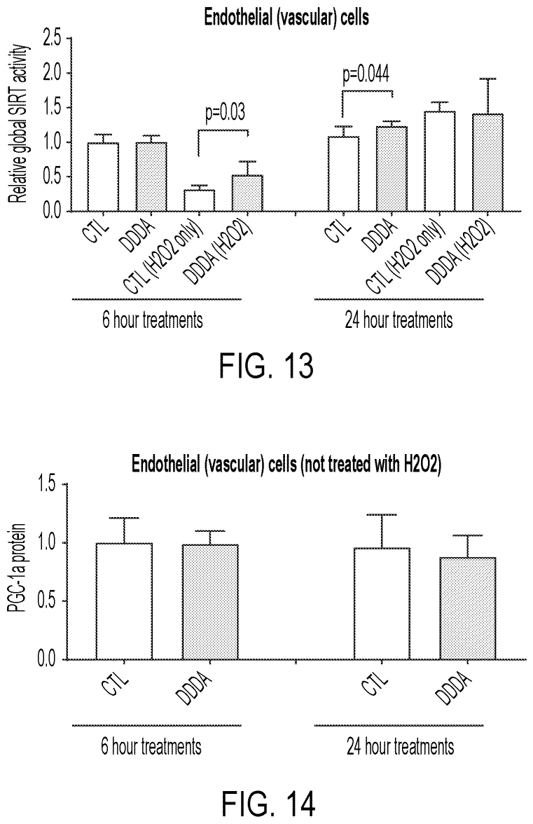

Global SIRT activity was measured in the endothelial cells and exemplary results are shown in FIG. 13. Here, the data demonstrate that after 6 hours of DDDA exposure (in the presence of hydrogen peroxide) and after 24 hours of DDDA exposure (without hydrogen peroxide) global SIRT activity was significantly (p=0.03 and p=0.044, respectively). These data once more confirm and reiterate what was found in liver cells, and warrants examining how DDDA affects SIRT activity in vivo. N=5-6 plates per treatment.

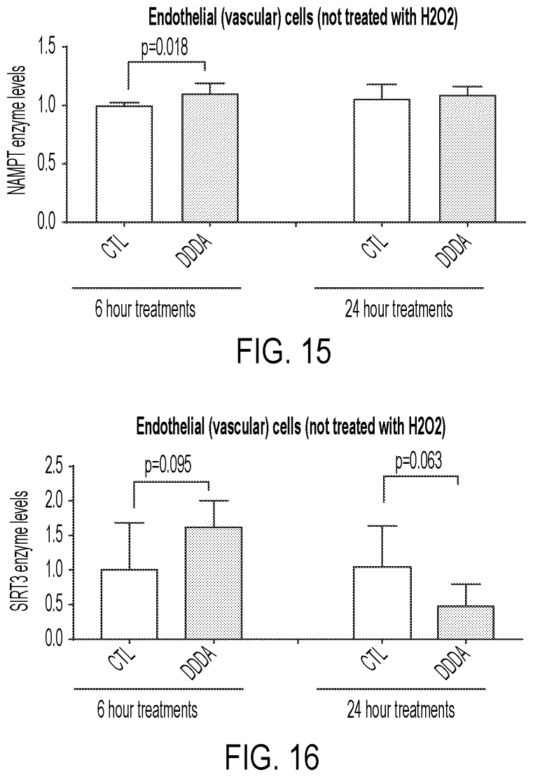

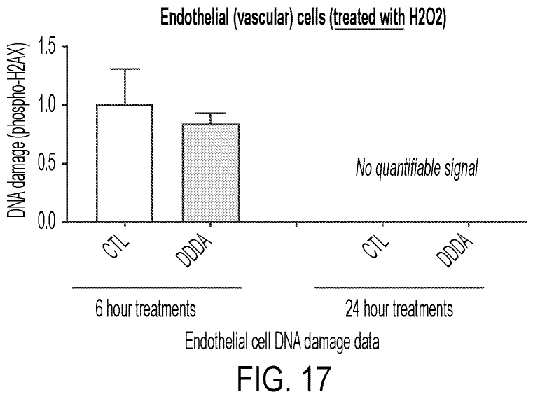

FIG. 14 shows exemplary results for PGC-1α. Notably, while PGC-1α was affected by exposure to DDDA, endothelial cells did not show a statistically significant difference (N=5-6 plates per treatment). On the other hand, where endothelial cells were exposed to DDDA, there was a significant difference in NAMPT enzyme levels as is exemplarily depicted in FIG. 15. Here, the data show that 6 hours of DDDA increased NAMPT enzyme levels (p=0.018; N=5-6 plates per treatment). Similar to hepatocytes, these data strongly suggest potential effects of DDDA on NAMPT enzyme regulation and NAD+ levels.

With respect to specific SIRT proteins, the inventors tested SIRT3, and exemplary results are shown in FIG. 16. Here, the exposure to DDDA caused a strong and acute increase in SIRT3 that was followed at 24 hours by a significant decline, suggesting tight regulation of SIRT3 expression. Notwithstanding, the 6-hour data here may explain why the up-regulation was observed in 24-hour DDDA-treated cells with global SIRT activity in FIG. 13 (p=0.044). N=5-6 plates per treatment.

Finally, the inventors also evaluated DNA damage in endothelial cells after oxidative insult by H₂O₂, and exemplary results are shown in FIG. 17. These data show that 6 hours of DDDA numerically decreased DNA damage, although the p-value was >0.100. These data agree in principle with the antioxidant data in endothelial cells as well as the DNA damage and antioxidant data in liver cells. N=5-6 plates per treatment.

Based on their beneficial safety and solubility profile and their significant antioxidant potential, the inventors further hypothesized that oral administration of dodecanedioc acid (e.g., as the sodium salt) could prevent and reverse NASH onset by reducing hepatic oxidative stress. To this end, the inventors performed two separate studies in rats to understand if dodecanedioic acid (DODA) could protect from (Study 1) and/or reverse (Study 2) NASH. In the first study DODA was administered together with a high-fat high-cholesterol diet for 4 weeks, while in the second study rats were first fed high-fat high-cholesterol diet for 9 weeks to induce NASH and then DODA was administered for 4 weeks to assess its possible reversal. In both studies, NASH presence was confirmed by hepatic histology. Furthermore, using primary hepatocytes and HSCs the inventors investigated the role of DODA in hepatic stellate cells activation.

### Animal Studies

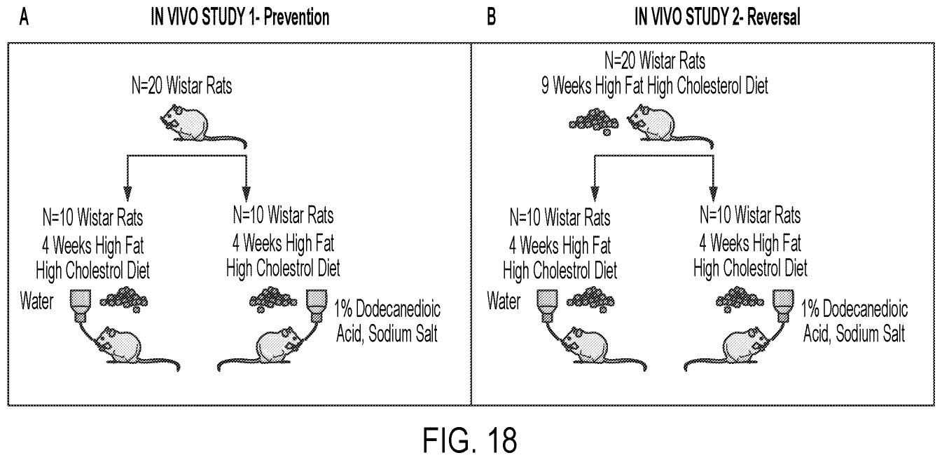

Study design: All animal procedures were approved by the Catholic University of Rome Institutional Animal Care Committee. The design of the studies is summarized in FIG. 18, Panels A and B.
Study 1: Twenty adult Wistar rats, aged 8-10 weeks, were included in the study. The rats were housed in individual cages at 22° C. with 12-h light cycles and had ad libitum access to food and water. Rats were fed a high-fat high-cholesterol diet (44% Carbohydrate, 14% Protein, 42% fat+0.2% Cholesterol) (Mucedola, Milan, IT) for four weeks in the presence or absence of 1% Dodecanedioic acid, sodium salt (w/vol) dissolved in the water (FIG. 18, Panel A). Body weight and food/water intake were monitored weekly.
Study 2: Twenty adult Wistar rats, aged 8-10 weeks, were included in the study. The rats were housed in individual cages at 22° C. with 12-h light cycles and had ad libitum access to food and water. Rats were fed a high-fat high-cholesterol diet (44% Carbohydrate, 14% Protein, 42% fat+0.2% Cholesterol) (Mucedola, Milan, IT). After nine weeks of diet, rats were randomly assigned to one of the following groups: Four weeks of high-fat high-cholesterol diet or four weeks of high-fat high-cholesterol diet and 1% Dodecanedioic acid, sodium salt (w/v) dissolved in the water FIG. 18, Panel B). Body weight and food/water intake were monitored weekly.

Oral Glucose Test Tolerance (OGTT): All animals underwent an OGTT at the end of the study. After an overnight fasting, the rats received a 50% D-glucose solution (1 g/kg body weight) by oral gavage. Blood samples were taken by tail bleeding and collected in EDTA tubes. All blood samples were immediately centrifuged, and plasma divided into appropriate subsamples and stored at −20° C. for further analysis. Blood glucose and was measured at 0, 20, 40, 60, 80, 100 and 120 minutes, while plasma insulin at 0, 60 and 120 minutes. Blood glucose levels were measured by glucometer (Accu-Chek, Roche Diagnostics Division, Grenzacherstrasse, CH). Plasma insulin was measured by ELISA (EMD Millipore Corporation, Billerica, MA), with a sensitivity of 0.1 ng/ml and an intra-and inter-assay precision of 1.9% and 7.6%, respectively.

Histology: The day of the sacrifice fresh portions of liver were cut, embedded in cryo-embedding media (OCT) and snap frozen in liquid nitrogen. Biopsies were cut using a cryostat (5 μm) and slides stored a −20° C. until analyses.

Hematoxylin and Eosin staining was performed to assess hepatic steatosis. Slides were fixed 10 minutes with 95% ethanol, stained with hematoxylin for 1 minute, washed with distilled water, stained with eosin for 30 seconds and cleared in two changes of pure ethanol and two changes of xylene.

Oil Red O was performed to assess intracellular lipid accumulation. Slides were fixed overnight with 4% formalin, stained with Oil Red O solution for 1 hour. Counterstain was performed with Hematoxylin solution.

Sirius Red was used to identify hepatic fibrosis. Slides were fixed 10 minutes with 4% formalin, stained in Direct red 80 for 1 hour, washed in acidified water and dehydrate in 3 changes of absolute ethanol. After brief clearing in xylene, the slides were mounted in a resinous medium. Images were taken with an optical microscope (Leica DM2000, Wetzlar, DE). All reagents for histological analysis were obtained from Sigma-Aldrich (St. Louis, MO).

### In Vitro Experiments

Primary hepatocytes: Hepatocytes were isolated by a two-step collagenase perfusion technique (6). Briefly, the inferior vena cava (IVC) was cannulated with a 24-gauge ¾-inch angiocatheter (BD) and the portal vein was cut. The liver was perfused via the IVC with 100 mL of Liver Perfusion Medium (Invitrogen) at 37° C., followed by perfusion with 100mL of collagenase type IV (Sigma-Aldrich, St. Louis, MO) in Hank's Balanced Salt Solution (HBSS, containing calcium and magnesium: GIBCO). After the liver was digested, it was dissected out and cut into small pieces and passed through a 100 μm strainer (Falcon). Hepatocytes were separated from non-parenchymal cells (NPCs) by low-speed centrifugation (50 g×5 mins), and further purified by Percoll gradient centrifugation (50% v/v, Sigma).

Nile Red: Cells were cultured for 24 hours in DMEM with 10% FBS. Prior stimulation, cells were incubated in serum free DMEM overnight. To assess lipid droplet accumulation, primary hepatocytes were stimulated for 24 hours with complete DMEM medium supplemented with Palmitic Acid (0.4 mM) and with or without 1% (w/vol) Dodecanedioic acid, sodium salt. After the stimulation, cells were stained with Nile Red (100 ng/ml) for 45 minutes. Stained cells were used for immunofluorescence to quantify the deposition of lipid droplets. Nuclear staining was performed with DAPI and images of lipid droplets were taken using confocal microscope Nikon A1 RHD25 and NIS-Elements imaging software was used to analyze images.

Palmitic acid treatment and preparation of conditioned medium: 100 mM palmitate stock solution was prepared in 0.1 mM NaOH by heating at 70° C. A 10% (w/v) FFA-free BSA (Sigma) solution was prepared in ddH2O and maintained at 55° C. in a water bath. 10 mM FFA/1% BSA solution was obtained by complexing the appropriate amount of palmitate stock solution to 10% BSA at 55° C. for another 30 min. The above solution was then cooled to 25° C., filter sterilized and stored at −20° C. until use. Primary hepatocytes were grown in DMEM supplemented with palmitate (0.4 mM). After 24 h. CM was clarified by centrifugation at 6000 g to remove cell debris, sterile filtered with a 0.45 μm pore size membrane filter and stored in aliquots at −20° C. until use.

Hepatic Stellate cells: Primary hepatic stellate cells were isolated from Wistar rats as described previously (7). In brief, the rats were surgically opened under anesthesia, and the livers were perfused through the IVC with 0.5 mM EGTA, after which the portal vein was cut and the upper IVC was clamped. After 2 min, the livers were perfused with a Pronase (Roche, Basilea, CH) solution for 5 min and then Collagenase-B (11088831001, Roche) solution for 7 min. The livers were removed and minced in a pronase/collagenase/DNAse-I (Roche, Basilea, CH) solution for 24 min and then passed through a 70-μm cell strainer. Liver cells were centrifuged and washed twice in Gey's balanced salt solution (GBSS) followed by density gradient separation of HSCs using a Histodenz (Sigma-Aldrich, St. Louis, MO) solution. After centrifugation. HSCs that were in the interface were collected. Cells were cultured for 48 hours in DMEM with 10% FBS. To induce fibrogenic activation and mitochondrial oxidative stress, primary HSCs were incubated with conditioned media obtained from steatotic primary hepatocytes for 3 days (8). HSCs activation was assessed by flow cytometry by evaluating alpha-smooth muscle Actin (α-SMA) and Collagen type I alpha 1 (COLIA1) protein expression. α-SMA and COLIA1 antibodies were obtained from Thermo Fisher scientific (Waltham, MA). Flow cytometric analysis was conducted with CytoFlex (Beckman Coulter, Brea, CA) and data analyzed with Kaluza software (Beckman Coulter, Brea, CA).

Statistical Analysis: The inventors used nonparametric tests because of the relatively small number of animals and because many variables were not normally distributed. To compare outcomes between the two groups the inventors used the Mann-Whitney U test. Repeated-measures ANOVA was used to compare blood glucose and plasma levels of insulin during OGTT. Data are expressed as the mean±SEM unless otherwise specified. Statistical significance was set at P<0.05 (two-tailed).

### Results

DODA administration prevents the onset of insulin resistance and NASH: To test the hypothesis that DODA could prevent the development of diet-induced insulin resistance and NASH, the inventors administered 1% (w/vol) of DODA in the drinking water together with a high fat-high cholesterol diet for 4 weeks. Rats fed high fat-high cholesterol diet without the administration of DODA were included as controls.

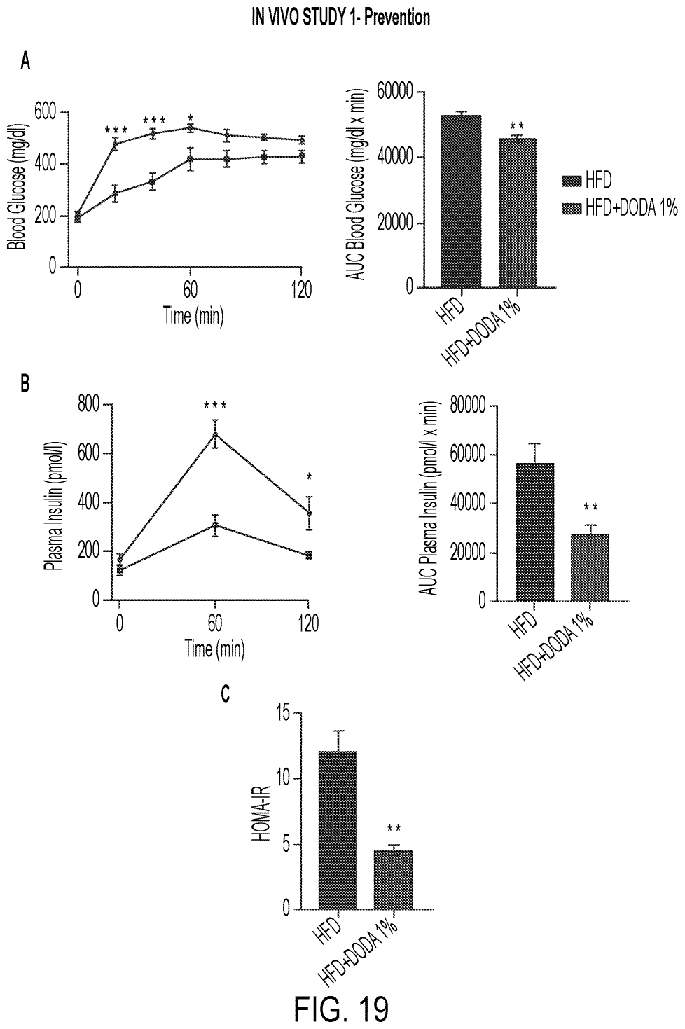

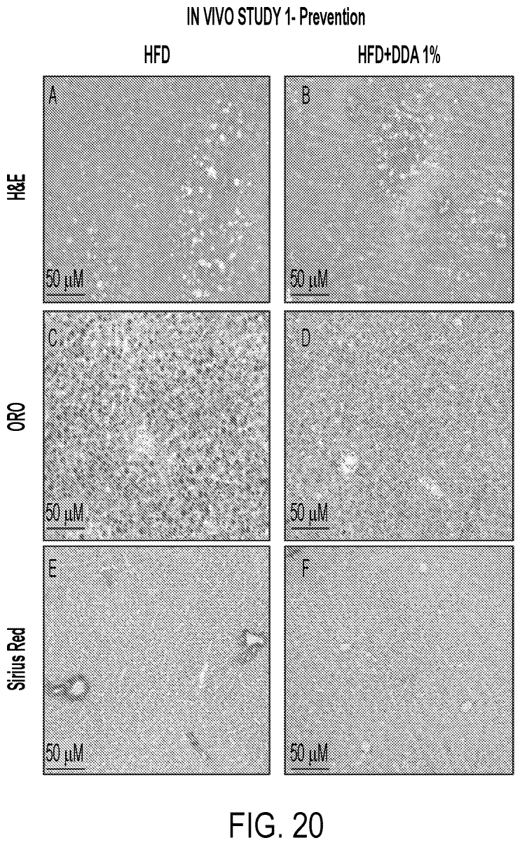

When compared to the controls, rats treated with DODA showed a decrease in blood glucose and plasma insulin concentrations during an oral glucose tolerance test (FIG. 19, Panels A and B). Consistently, hepatic insulin resistance, assessed by HOMA-IR, was decreased in the DODA group (FIG. 19, Panel C). Histological analysis for hepatic hallmarks of NASH showed that DODA treatment reduced hepatic steatosis, neutral lipids deposition and fibrosis (FIG. 20, Panels A-F).

Taken together these data suggest that DODA treatment was able to prevent diet-induced insulin resistance and NASH.

DODA administration reverses insulin resistance and NASH: To assess if DODA could reverse the development of diet-induced insulin resistance and NASH, rats were first fed high fat high cholesterol diet for 9 weeks to develop insulin resistance and NASH. After 9 weeks of diet, DODA was administered for 4 weeks to assess insulin resistance and NASH reversal. Rats fed high fat high cholesterol diet without the administration of DODA were included as controls.

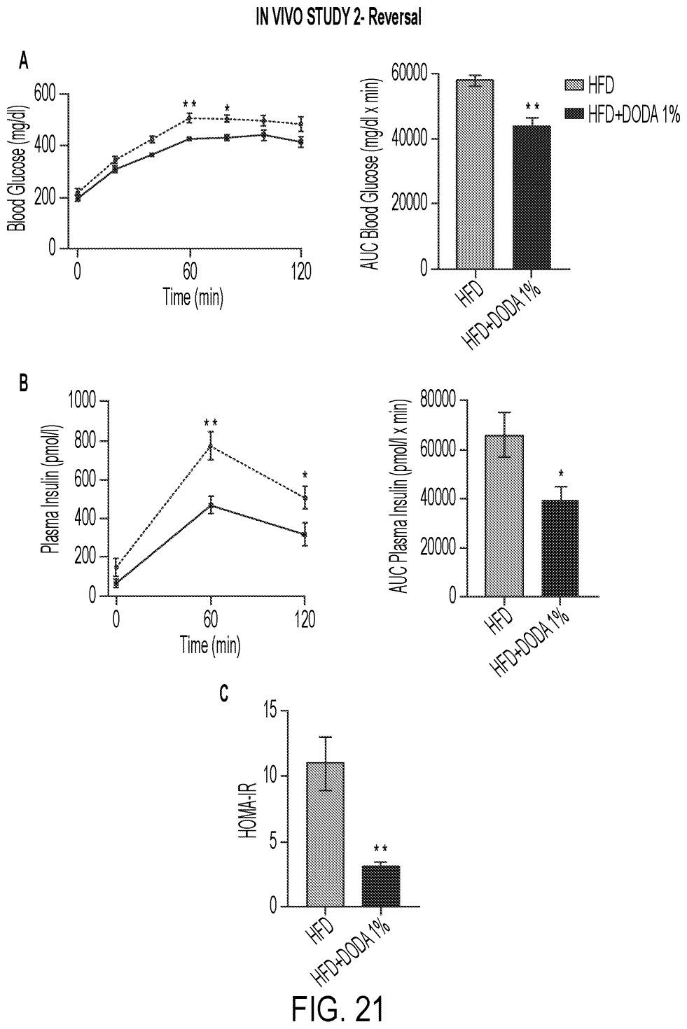

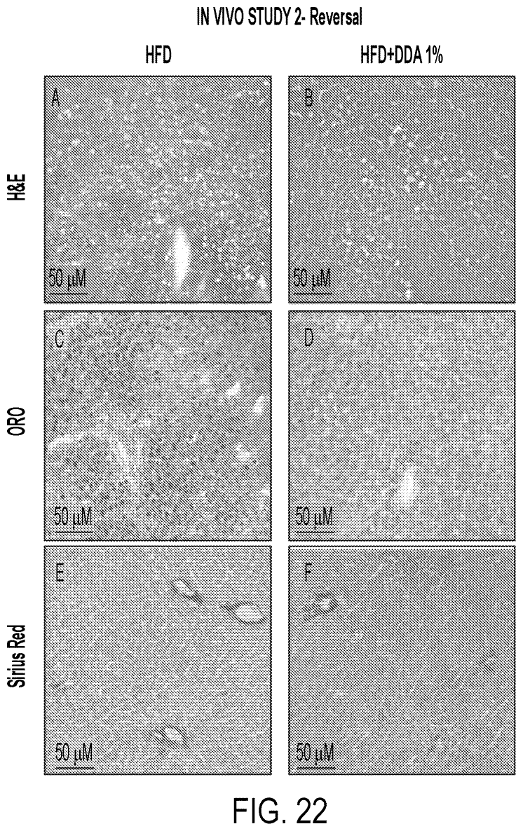

DODA treatment caused a decrease of blood glucose and plasma insulin concentrations during an oral glucose tolerance test (FIG. 21, Panels A and B) and reverse insulin resistance as assessed by HOMA-IR (FIG. 21, Panel C). Histological analysis revealed that DODA treatment reverse the effect of diet-induced NASH by decreasing hepatic steatosis, neutral lipids and fibrosis (FIG. 22, Panels A-F).

These results indicated that DODA treatment could reverse both diet-induced insulin resistance and NASH.

DODA induces HSCs apoptosis contributing to the resolution of liver fibrosis: Previous studies have demonstrated that the reversal of liver fibrosis leads to a reduction of myofibroblasts, and that this reduction is associated with increased myofibroblast apoptosis. Moreover, inducing myofibroblast apoptosis by pharmacological approaches accelerates fibrosis resolution suggesting a causative contribution of apoptosis to this process.

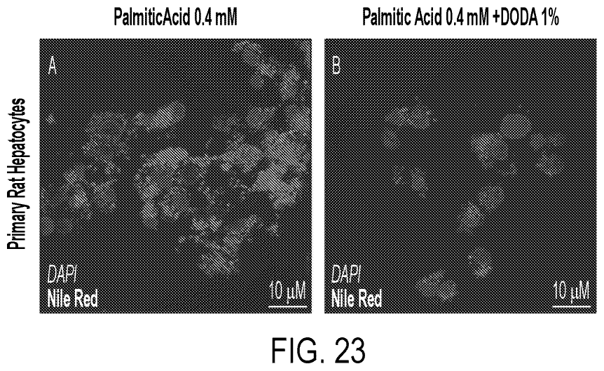

To explore the effects of DODA treatment on HSCs, the main fibrogenic cell type of the liver, the inventors used conditioned medium (CM) of steatotic hepatocytes to induce HSCs activation with or without 1% (w/vol) of DODA. Two days after isolation, rat primary HSCs were exposed for 3 days to CM from palmitate-treated hepatocytes and then were incubated with DODA for 24 hours. FIG. 23 depicts exemplary results for shows in vitro lipid droplets accumulation in primary hepatocytes by Nile red staining of palmitate treated (0.4 mM) primary hepatocytes incubated with or without DODA 1% (w/vol).

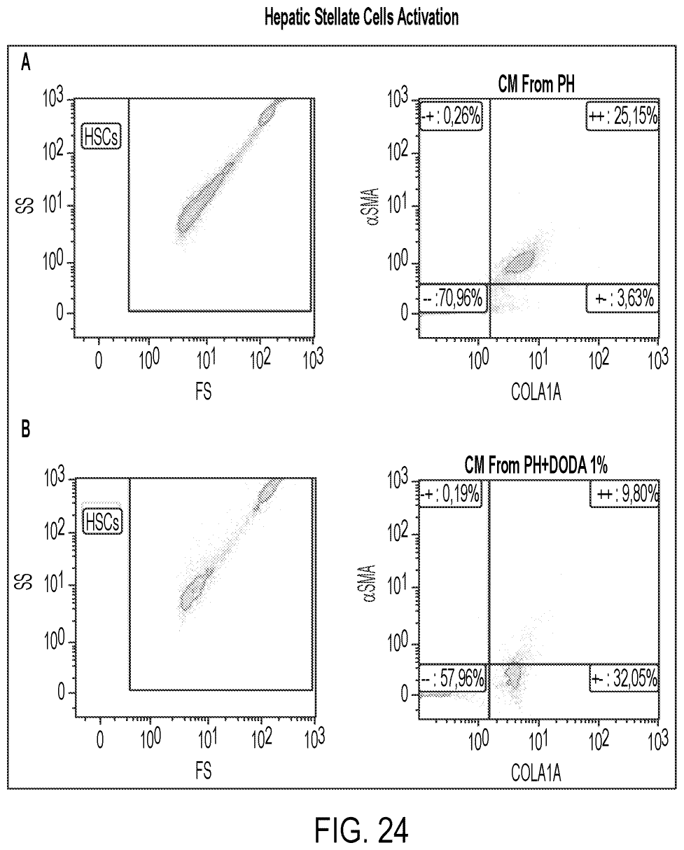

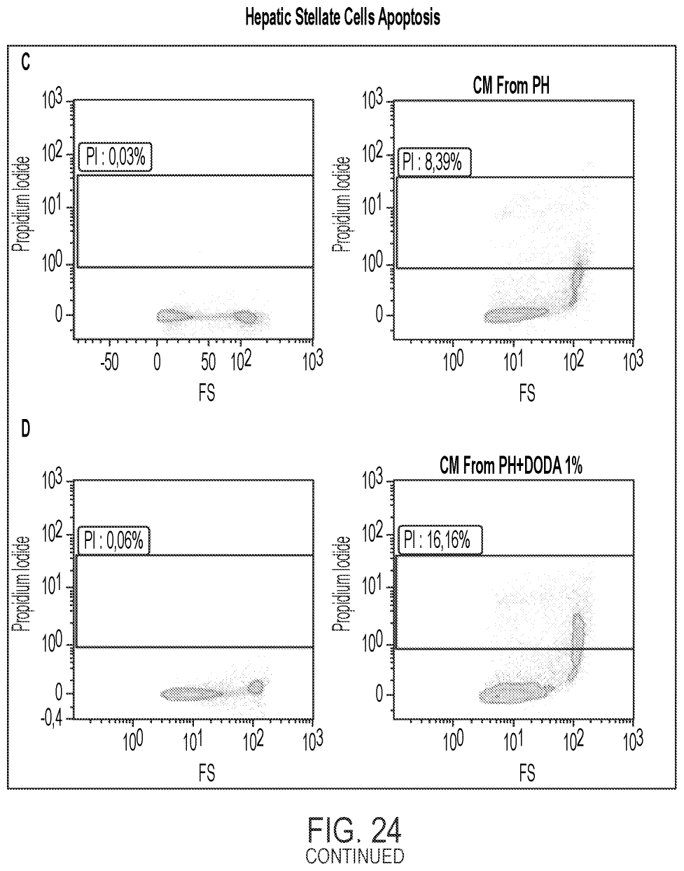

Subsequently, protein expression of two established markers of HSCs activation, namely collagen type 1 (COLA1A) and alpha-smooth muscle actin (αSMA), was determined by flow cytometry analysis. HSCs exposed to CM from steatotic hepatocytes in combination with DODA revealed a lower expression of COLA1A and α-SMA (9.18% vs. 25.15%) when compared to HSCs incubated without DODA (FIG. 24, Panels A and B). Next, the inventors analyzed the effect of DODA on HSCs apoptosis. HSCs treated with CM from steatotic hepatocytes together with DODA showed an increase in cell death (16.16% vs. 8.39%) assessed by propidium iodide staining using flow cytometry (FIG. 24, Panels C and D).

Further tests can be performed to evaluate the effect of contemplated compounds and compositions on metabolism, gut microbiome, and aging as further detailed below:

Metabolism: Contemplated compositions can be administered to a healthy subject or a subject suffering from or diagnosed with a metabolic disorder such as dyslipidemia, loss of insulin sensitivity, prediabetes, type 2 diabetes, obesity, fatty liver (NASH). It should also be noted that such subjects may have normal average daily caloric intake or have dietary behavior characteristic for overnutrition (see e.g., J Gerontol A Biol Sci Med Sci. 2021 Sep. 13;76(10):1714-1725). Consequently, one or more of the following biomarkers will be followed on an acute (e.g., T0, 30 min, 60 min, 90 min, 120 min, 180 min, 240 min) and long-term (e.g., T0, 1 day, 3 day, 7 day, 14 day, 21 day, 30 day) basis: fasting/average, post-prandial blood glucose and insulin levels, HbA1c, HOMA-1R, triglycerides, total/HDL/oxLDL/LDL cholesterol, lipid particle size, homocysteine, FGF21 expression, SIRT1 expression, body weight, BMI, visceral fat, serum levels of ALT, AST, GST, BUN, creatinine, ketones, and blood pressure.

Gut microbiome: The effect of contemplated compounds can be evaluated in vitro using the Simulator of the Human Intestinal Microbial Ecosystem (SHIME: see e.g., Chapter 27 in “The Impact of Food Bioactives on Health: in vitro and ex vivo models” by Verhoeckx K, Cotter P, López-Expósito I, et al., editors. Cham (CH): Springer; 2015). Such in vitro model advantageously reduces the impact of other physiological processes that might interfere with the effect of the polyamines on the microbial flora. However, where observations for in vivo conditions are preferred, it should be appreciated that the microbiome can be followed using fecal sampling and 16S rRNA sequencing as is well known in the art. Therefore, microbiome testing will reveal shifts in microbial species/families/orders (e.g., increase in abundance of Firmicutes decrease in abundance of Bacteroides) as well as a change in microbial overall diversity. Moreover, additional parameters that can be obtained from such in vivo and in vitro testing especially include quantification of short chain fatty acids (SCFA), butyrate-producing or bifidobacteria-producing bacteria, and ketone bodies such as (hydroxy) butyric acid, acetate, acetoacetate, propionate in the gut.

The aging processes can also be quantitatively followed by measurements of various markers that are often associated with caloric restriction, and especially contemplated markers include Sirtuin 1 activation, inhibition of insulin/insulin growth factor signaling, etc. (see e.g., Cell. 2011 Sep. 2;146(5):682-95).

### Efficacy of Dodecanedioic Acid Treatment for NASH Prevention and Reversal

In the US alone, the number of NAFLD cases are expected to grow from 83 million in 2015 to 101 million in 2030, with 27% of cases meeting the criteria for NASH. The rising disease prevalence is accompanied by an increased number of individuals with both cirrhosis and end-stage liver disease, needing liver transplantation.

Nonalcoholic steatohepatitis (NASH) is a pro-inflammatory state that leads to the activation of hepatocytes, Kupffer cells (KCs) and hepatic stellate cells (HSCs). Although the exact cause of NAFLD is not defined, recent studies have shown that increased rates of hepatic mitochondrial oxidation could lead to increased reactive oxygen species (ROS) formation and promote the progression from NAFLD to NASH, by activating hepatic stellate cells (HSCs). ROS-activated HSC undergo a phenotypic switch and deposit an excessive amount of extracellular matrix that alters the normal liver architecture and leads to liver fibrosis.

Hepatic fat accumulation, the histological hallmark of NASH, is often associated with increased fatty acid delivery, intensified de novo lipogenesis (DNL), and decreased lipid disposition via fatty acid oxidation. Several clinical studies indicated that the rate of DNL is increased in NASH and is responsible for around 20-30% of fat accumulation in the liver while in the physiological state is around 5-10%, underlying the indispensable role of DNL in the progression of NASH.

Cytosolic citrate, a key precursor and regulator for de novo fatty acid synthesis, has been considered an important metabolite that links glucose and lipid metabolism. Indeed, citrate inhibits phosphofructokinase (PFK), thereby reducing glycolytic flux and promotes the polymerization and thus the activation of acetyl-CoA carboxylase (ACC) which catalyzes the rate limiting step in DNL. The Na+-coupled dicarboxy late transporter from the SLC13 family, NaCT (gene SLC13A5) mediates the transport of citrate into cells and plays an important role in determining cytosolic citrate concentrations.

SLC13a5 knockout (KO) mice show improvements in glycemic control which can be attributed to the suppression of glucose production. Additionally, SLC13a5 KO mice that have been fed a high fat diet (HFD) display a reduction in body weight and hepatic lipid concentrations compared to their wild type counterparts. Moreover, dicarboxylates may be capable of inhibiting NaCT-mediated cellular uptake of citrate in vitro and in vivo. Thus, the inhibition of NaCT may be a beneficial strategy for treating metabolic disorders and NASH.

Currently, there are no FDA approved pharmacological treatments for NAFLD. Recently. dicarboxylic acids (DAs) have garnered much interest in their potential health benefits due to their wide range of possible therapeutic actions. Unlike the homologous fatty acids, DAs are soluble in water as salts and have been proved to be safe in both experimental animals and humans. Dodecanedioic acid (referred to herein as C12 or DDDA, which terms are used interchangeably throughout the disclosure), belonging to the family of straight-chain dicarboxylic acids, is β-oxidized to CO2 and H2O via formation of acetyl-CoA and succinyl-CoA. The inventors investigated if the oral administration of Dodecanedioc acid, Sodium Salt could prevent and/or reverse NASH onset. The inventors contemplated that C12 could inhibit NaCT reducing citrate uptake and thereby attenuates ectopic fat accumulation in the liver.

To this end, the inventors performed two separate study, in rats, to understand if C12 could protect (Study 1) and/or reverse (Study 2) NASH. In the first study C12 was administered together with a high fat-high cholesterol diet for 4 weeks, while in the second study rats were first fed high fat-high cholesterol diet for 9 weeks to develop NASH and then C12 was administer for 4 weeks to assess its reversal. In both studies, NASH presence was assessed by hepatic histology. Finally, the inventors also performed two in vitro study to investigate the role of C12 in primary human hepatic stellate cells activation and to understand if C12 could reduce citrate uptake in primary human hepatocytes by lowering the expression of citrate transporter NaCT.

### Methods

### Animal Study, Study Design

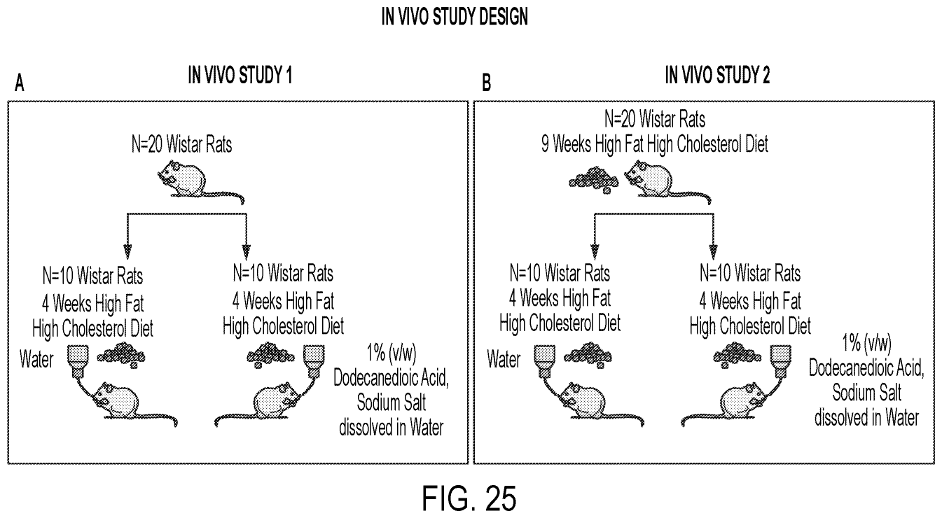

The design of the studies is summarized in FIG. 25. Panels A and B.
Study 1: Twenty adult Wistar rats, aged 8-10 weeks, were included in the study. The rats were housed in individual cages at 22° C. with 12-h light cycles and had ad libitum access to food and water. Rats were fed a high fat high cholesterol diet (44% Carbohydrate, 14% Protein, 42% fat+0.2% Cholesterol) for 4 weeks in the presence or absence of 1% Dodecanedioic acid, sodium salt dissolved in the water (FIG. 25, Panel A). Body weight and food/water intake were monitored weekly.
Study 2: Twenty adult Wistar rats, aged 8-10 weeks, were included in the study. The rats were housed in individual cages at 22° C. with 12-h light cycles and had ad libitum access to food and water. Rats were fed a high fat-high cholesterol diet (44% Carbohydrate, 14% Protein, 42% fat+0.2% Cholesterol). After 9 weeks of diet, rats were randomly assigned to one of the following groups: 4 weeks of high fat-high cholesterol diet or 4 weeks of high fat-high cholesterol diet and 1% Dodecanedioic acid, sodium salt dissolved in the water (FIG. 25, Panel B). Body weight and food/water intake were monitored weekly.

Oral Glucose Test Tolerance (OGTT): All animals underwent an OGTT at the end of the study. After an overnight fasting, all rats received a 50% D-glucose solution (1 g/kg body weight) by oral gavage. Blood samples were taken by tail bleeding and collected in EDTA tubes. All blood samples were immediately centrifuged, and plasma divided into appropriate subsamples and stored at −20° C. for further analysis. Blood glucose and plasma insulin were measured at 0, 20, 40, 60, 80, 100 and 120 minutes. Blood glucose levels were measured by glucometer. Plasma insulin was measured by ELISA, with a sensitivity of 0.1 ng/ml and an intra-and inter-assay precision of 1.9% and 7.6%, respectively.

Histology: The day of the sacrifice fresh portions of liver were cut, embedded in cryo-embedding media (OCT) and snap frozen in liquid nitrogen. Biopsies were cut using a cryostat (5 μm) and slides stored a −20° C. until analyses.

Hematoxylin and Eosin staining was performed to assess hepatic steatosis. Slides were fixed 10 minutes with 95% ethanol, stained with hematoxylin for 1 minute, washed with distilled water, stained with eosin for 30 seconds and cleared in two changes of pure ethanol and two changes of xylene.

Oil Red O was performed to assess intracellular lipid accumulation. Slides were fixed overnight with 4% formalin. stained with Oil Red O solution for 1 hour. Counterstain was performed with Hematoxylin solution.

Sirius Red was used to identify hepatic fibrosis. Slides were fixed 10 minutes with 4% formalin, stained in Direct red 80 for 1 hour, washed in acidified water and dehydrate in 3changes of absolute ethanol. After brief clearing in xylene, the slides were mounted in a resinous medium. Images were taken with an optical microscope. All reagents for histological analysis were obtained from Sigma-Aldrich.

### Quantitative Real-Time PCR Analysis

Total RNA from human primary hepatocytes was extracted using the RNeasy Plus Mini Kit according to the indications provided by the company. A small aliquot of total RNA obtained (3 μl) was subjected to qualitative and quantitative control by using the microdrop. The qualitative and quantitative assessment of the individual samples was determined using a dedicated software. The total RNA was reverse transcribed into cDNA by using iScript RT. SYBR Green gene expression assays were performed according to the manufacturer's instruction using the iQTMSYBR Green Supermix and the CFX96 Touch Real-Time PCR Detection System. For this purpose, the following pairs of primer were used: solute carrier family 13 member 5 (SLC13A5) (forward 5′ AGAGGCAGTGGTAGTCGTGT 3′ (SEQ ID NO:1) and reverse 5′ TCCCCTTTAGCCCTTGTTCC 3′ (SEQ ID NO:2)), Diacylglycerol O-Acyltransferase 1 (DGAT1) (forward 5′ CTACAGGGACTGGTGGAATGC 3′ (SEQ ID NO:3) and reverse 5′ AGCAGGAGTAGGCCCCATAG 3′ (SEQ ID NO:4)), Fatty Acid Synthase (FASN) (forward 5′ GAATCCGCACAGGCTACCAA 3′ (SEQ ID NO:5) and reverse 5′ CTGGGCTTCACCATCACCAT 3′ (SEQ ID NO:6)), carnitine palmitoyltransferase 1A (CPT1A) (forward 5′ TGGGGAAGAGACAGACACCA 3′ (SEQ ID NO:7) and reverse 5′ ATCGTGGTAGAGCCAGACCT 3′ (SEQ ID NO:8)) and Acetyl-CoA carboxylase (ACC1) (forward 5′ CCACAACTACCATCACGCCT 3′ (SEQ ID NO:9) and reverse 5′ CAGGAACTCAGAAGCCCAGAA 3′ (SEQ ID NO:10)). mRNA expression levels were normalized to B2-microglobulin (forward 5′AGGACTGGTCTTTCTATCTCTTGT 3′ (SEQ ID NO:11); and reverse 5′ACCTCCATGATGCTGCTTACA 3′ (SEQ ID NO:12)) and quantification of relative gene expression, presented as percentage of the relevant baseline, was calculated using the 2-ACT (comparative threshold) method.

### In Vitro Experiments

Isolation of primary rat hepatocytes: Hepatocytes were isolated by a two-step collagenase perfusion technique. Briefly, the inferior vena cava (IVC) was cannulated with a 24-gauge ¾-inch angiocatheter and the portal vein was cut. The liver was perfused via the IVC with 100 mL of Liver Perfusion Medium at 37° C., followed by perfusion with 100 mL of collagenase type IV in Hank's Balanced Salt Solution (HBSS, containing calcium and magnesium: GIBCO). After the liver was digested, it was dissected out and cut into small pieces and passed through a 100 μm strainer (Falcon). Hepatocytes were separated from non-parenchymal cells (NPCs) by low-speed centrifugation (50 g×5 mins), and further purified by Percoll gradient centrifugation (50% v/v, Sigma).

Nile Red: Cells were cultured for 24 hours in DMEM with 10% FBS. Prior stimulation, cells were incubated in serum free DMEM overnight. To assess lipid droplet accumulation, primary hepatocytes were stimulated for 24 hours with complete DMEM medium supplemented with Palmitic Acid (0.4 mM) and with or without 1% (w/vol) Dodecanedioic acid, sodium salt. After the stimulation, cells were stained with Nile Red (100 ng/ml) for 45 minutes. Stained cells were used for immunofluorescence to quantify the deposition of lipid droplets. Nuclear staining was performed with DAPI and images of lipid droplets were taken using confocal microscope Nikon A1 RHD25 and NIS-Elements imaging software was used to analyze images.

Palmitic acid treatment and preparation of conditioned medium: 100 mM palmitate stock solution was prepared in 0.1 mM NaOH by heating at 70° C. A 10% (w/v) FFA-free BSA solution was prepared in ddH2O and maintained at 55° C. in a water bath. 10 mM FFA/1% BSA solution was obtained by complexing the appropriate amount of palmitate stock solution to 10% BSA at 55° C. for another 30 min. The above solution was then cooled to 25° C., filter sterilized and stored at −20° C. until use.

Primary rat hepatocytes were grown in DMEM supplemented with palmitate (0.4 mM). FFA-free-BSA-treated cells (0.4% w/v) served as controls. After 24 h, CM was clarified by centrifugation at 6 000 g to remove cell debris, sterile filtered with a 0.45 μm pore size membrane filter and stored in aliquots at −20° C. until use.

Isolation of primary rat hepatic stellate cells: Primary hepatic stellate cells were isolated from Wistar rats. The rats were surgically opened under anesthesia, and the livers were perfused through the inferior vena cava (IVC) with 0.5 mM EGTA, after which the portal vein was cut and the upper IVC was clamped. After 2 min, the livers were perfused with a Pronase solution for 5 min and then Collagenase-B solution for 7 min. The livers were removed and minced in a pronase/collagenase/DNAse-I (10104159001, Roche) solution for 24 min and then passed through a 70-μm cell strainer. Liver cells were centrifuged and washed twice in Gey's balanced salt solution (GBSS) followed by density gradient separation of HSCs using a Histodenz solution. After centrifugation, HSCs that were in the interface were collected. Cells were cultured for 48 hours in DMEM with 10% FBS. To induce fibrogenic activation and mitochondrial oxidative stress, primary HSCs were incubated with conditioned media obtained from steatotic primary hepatocytes for 3 days.

HSCs activation was assessed by flow cytometry by evaluating alpha-smooth muscle Actin (α-SMA) and Collagen type I alpha 1 (COLIA1) protein expression. α-SMA and COLIA1 antibodies were obtained from Thermo Fisher scientific. Flow cytometric analysis was conducted with CytoFlex and data analyzed with Kaluza software.

Isolation of human primary hepatocytes: Tissue obtained during percutaneous liver biopsy, was diced (<3 mm) and washed in HBSS to remove excess blood. Tissue was then transferred to a tube containing pre-warmed EGTA buffer (HBSS, 0.5 mM EGTA, 0.5% fatty acid free bovine serum albumin (BSA)) and agitated (100 rpm) in a water bath with shaking bed for 10 min, 37° C. After three washes, to remove the remaining blood and EGTA, the tissue was placed in pre-warmed digestion buffer (HBSS, 0.05% collagenase IV, 0.5% fatty acid free BSA, 10 mM CaC12) and agitated (100 rpm) in a water bath with shaking bed for 30 min, 37° C. The digested tissue was filtered through 100 μm cell strainer while the remaining tissue was again digested in fresh digestion buffer (20). Cell suspensions were pooled and centrifuged at 80 g for 5 min, 4° C. and the supernatant discarded. Cells were grown until confluent and used for citrate uptake analysis.

In vitro Citrate Uptake: Primary human hepatocytes were incubated with or without Dodecanedioic acid, sodium salt (0.1-0.25-0.5-1-1.5% w/vol) for 30 minutes. Following the incubation step, citrate was added at a final concentration of 150 μM for 40 minutes. At the end of the stimulation citrate uptake was assessed by a commercial Citrate Assay Kit and data analyzed with Varioskan LUX multimode microplate reader.

In vitro Palmitic Acid Stimulation: Primary human hepatocytes were stimulated with palmitic acid (0.4 mM) and with or without Dodecanedioic acid, sodium salt (1% w/vol) for 24 hours. At the end of the stimulation de novo lipogenesis, fatty acid beta-oxidation and SLC13A5 gene expression were assessed by Quantitative Real-Time PCR Analysis.

Deuterated water for de novo lipogenesis assessment: Cells were incubated for 24 h with palmitic acid (0.4 mM) and with or without Dodecanedioic acid, sodium salt (1% w/vol) for 24 hours. Cell cultured medium was enriched with 10% deuterated water to evaluate de novo lipid synthesis and gluconeogenesis. Cell extracts and medium samples were immediately freeze in nitrogen and stored at −80° C. for further analysis.

Statistical Analysis: Nonparametric tests were used because of the relatively small number of animals and because many variables were not normally distributed. To compare outcomes between the two groups we used the Mann-Whitney U test. Repeated-measures ANOVA was used to compare blood glucose and plasma levels of insulin during OGTT. Data are expressed as the mean±SEM unless otherwise specified. Statistical significance was set at P<0.05 (two-tailed).

### Results

C12 administration prevents the onset of insulin resistance and NASH: To test the hypothesis that C12 could prevent the development of diet-induced insulin resistance and NASH, we administered 1% (w/vol) of C12 in the drinking water together with a high fat-high cholesterol diet for 4 weeks. Rats fed high fat-high cholesterol diet without the administration of C12 were included as controls.

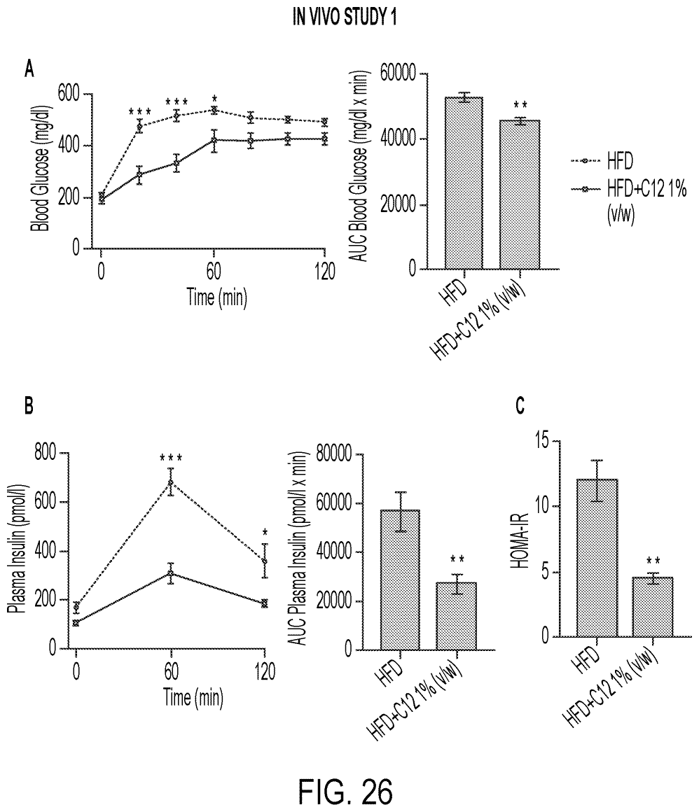

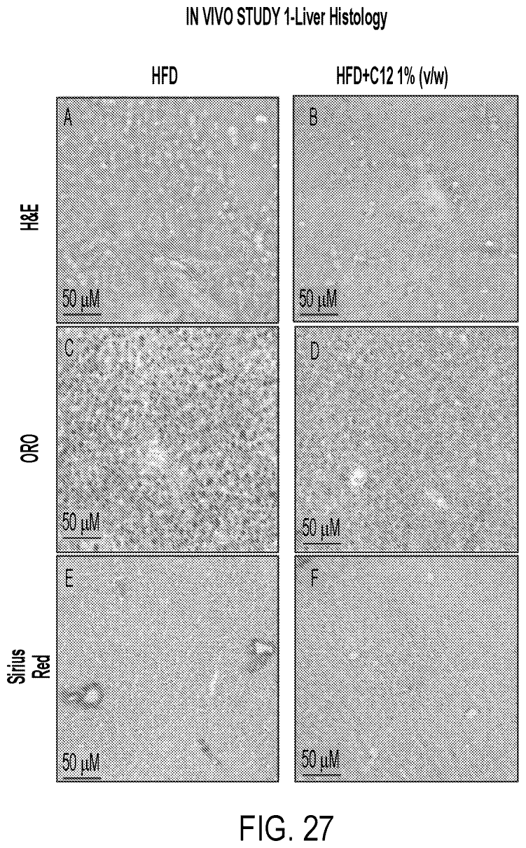

When compared to the controls, rats treated with C12 showed a decrease in blood glucose and plasma insulin concentrations during an oral glucose tolerance test (FIG. 26, Panels A and B). Consistently, hepatic insulin resistance, assessed by HOMA-IR, was decreased in the C12 group (FIG. 26, Panel C). Histological analysis for hepatic hallmarks of NASH showed that C12 treatment reduced hepatic steatosis, neutral lipids deposition and fibrosis (FIG. 27, Panels A-F). Taken together these data suggest that C12 treatment was able to prevent diet-induced insulin resistance and NASH.

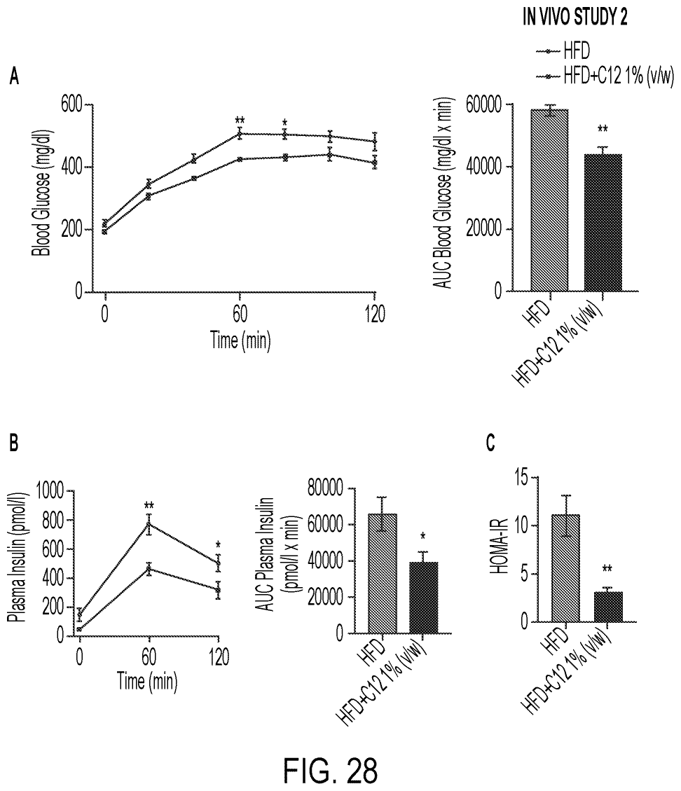

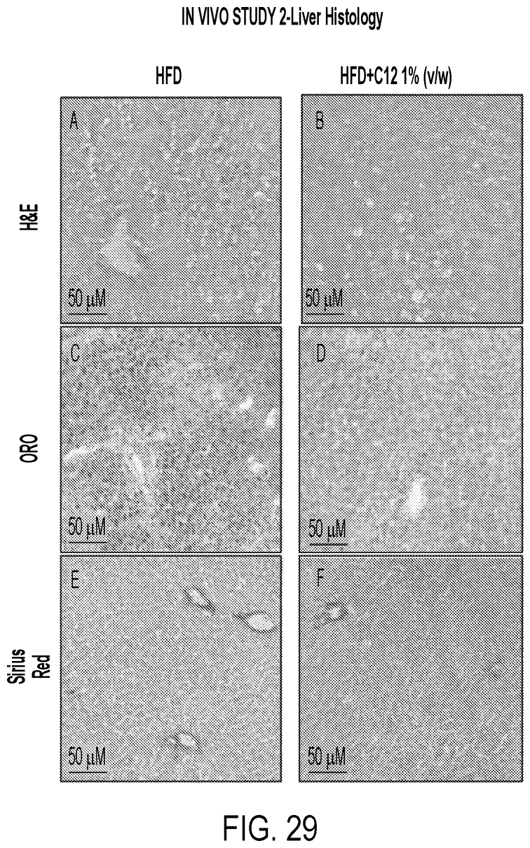

C12 administration reverses insulin resistance and NASH: To assess if C12 could reverse the development of diet-induced insulin resistance and NASH, rats were fed a high fat-high cholesterol diet for 9 weeks to develop insulin resistance and NASH. After 9 weeks of diet, C12 was administer for 4 weeks to assess insulin resistance and NASH reversal. Rats fed high fat-high cholesterol diet without the administration of C12 were included as controls. C12 treatment caused a decrease of blood glucose and plasma insulin concentrations during an oral glucose tolerance test (FIG. 28, Panels A and B) and reverse insulin resistance as assessed by HOMA-IR (FIG. 28, Panel C). Histological analysis revealed that C12 treatment counteract the effect of diet-induced NASH by decreasing hepatic steatosis, neutral lipids accumulation and fibrosis (FIG. 29. Panels A-F). These results indicated that C12 treatment could reverse both diet-induced insulin resistance and NASH.

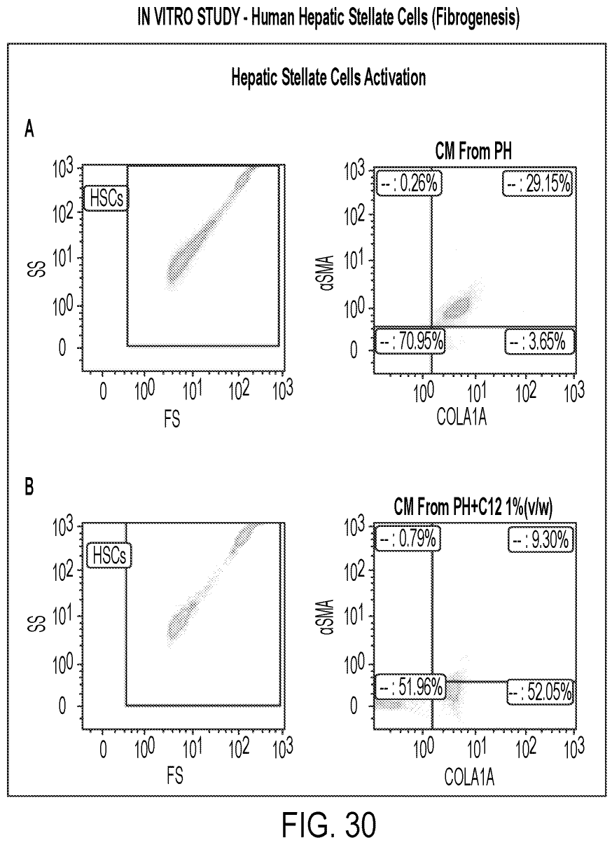

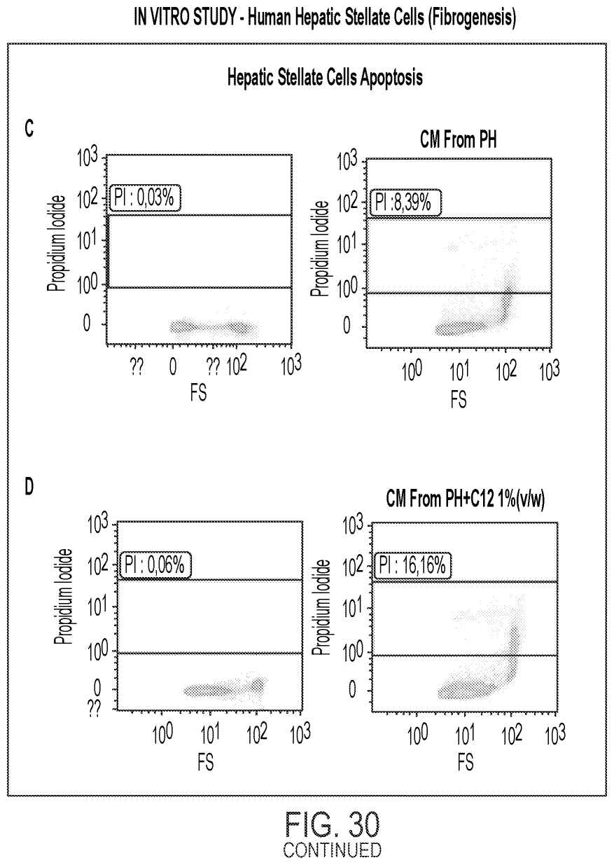

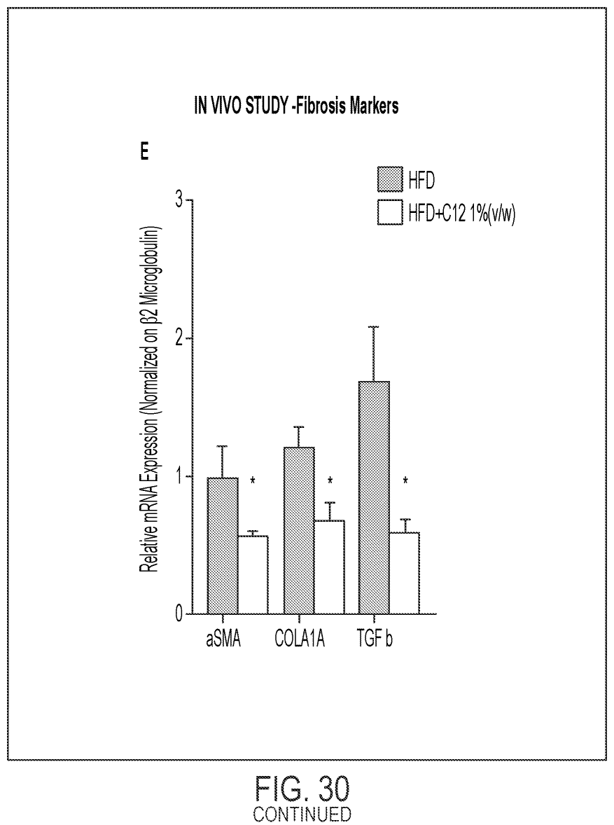

C12 induces HSCs apoptosis contributing to the resolution of liver fibrosis: Previous studies have demonstrated that the reversal of liver fibrosis leads to a reduction of myofibroblasts, and that this reduction is associated with increased myofibroblast apoptosis. Moreover, inducing myofibroblast apoptosis by pharmacological approaches accelerates fibrosis resolution suggesting a causative contribution of apoptosis to this process. To explore the effects of C12 treatment on HSCs, the main fibrogenic cell type of the liver, we used conditioned medium (CM) of steatotic rat primary hepatocytes to induce HSCs activation with or without 1% (w/vol) of C12. Two days after isolation, rat primary HSCs were exposed for 3 days to CM from palmitate-treated hepatocytes and then were incubated with C12 for 24 hours. Subsequently, protein expression of two established markers of HSCs activation, namely collagen type I (COLA1A) and alpha-smooth muscle actin (αSMA), was determined by flow cytometry analysis. HSCs exposed to CM from steatotic hepatocytes in combination with C12 revealed a lower expression of COLA1A and α-SMA (9.18% vs. 25.15%) when compared to HSCs incubated without C12 (FIG. 30, Panels A and B). Next, we analyzed the effect of C12 on HSCs apoptosis. HSCs treated with CM from steatotic hepatocytes together with C12 showed an increase in cell death (16.16% vs. 8.39%) assessed by flow cytometry using propidium iodide staining (FIG. 30, Panels C and D). Concomitantly, when compared to the control group, rats treated with C12 showed a decreased expression of the genes associated with fibrosis (αSMA, COLA1A and TGFβ) (FIG. 30. Panel E)

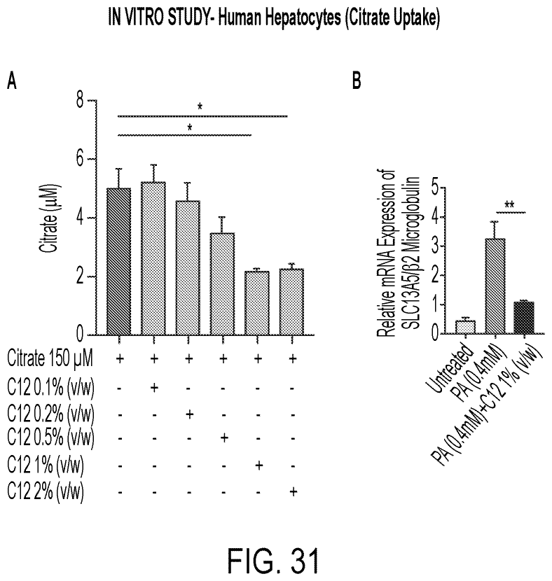

C12 inhibits citrate uptake reducing gene expression of citrate transporter SLC13A5: To test the hypothesis that C12 could inhibits citrate uptake human primary hepatocytes were incubated with or without C12 (0.1-1.5% v/w) and a fixed concentration of citrate (150 μM). Cells treated with C12 at 1 and 1.5% (v/w) showed a significant decrease of citrate uptake when compared with untreated cells (FIG. 31, Panel A). Moreover, human hepatocytes stimulated with C12 1% (v/w) in combination with palmitic acids (0.4 mM) showed a significant decrease in the gene expression of citrate transporter SLC13A5 when compared with cells treated with palmitic acid (0.4 mM) alone (FIG. 31, Panel B).

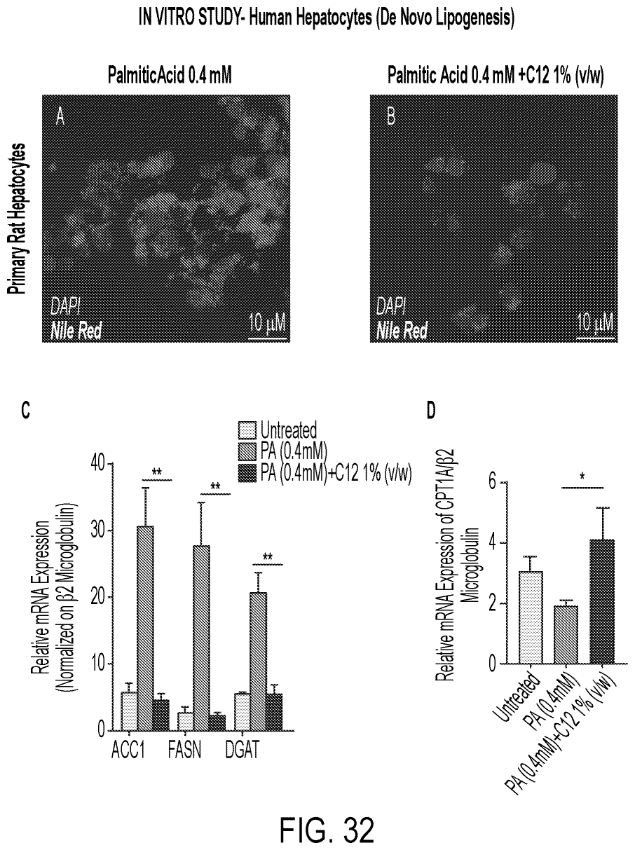

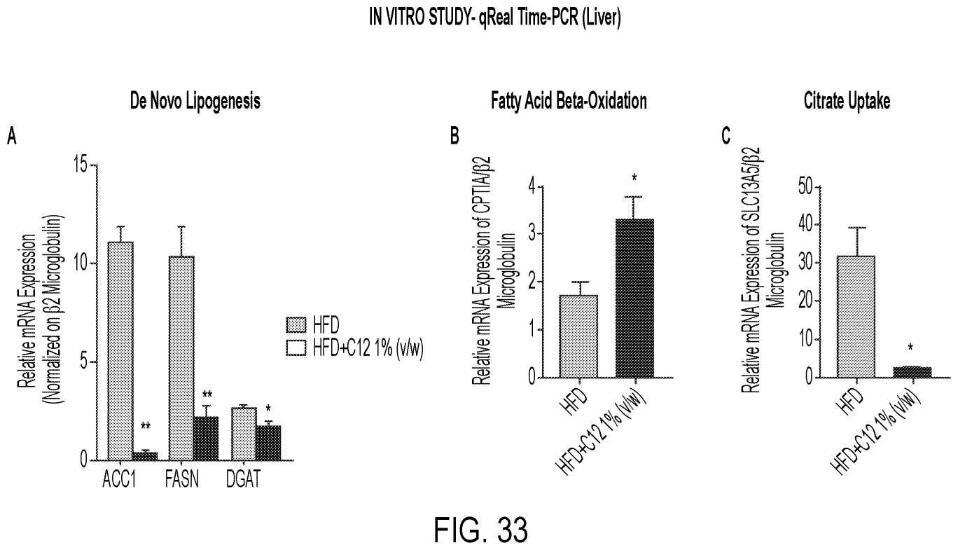

C12 decrease de novo lipogenesis and an increase in fatty acid oxidation in vitro and in vivo: To test the hypothesis that C12 could affect de novo lipogenesis and fatty acid beta-oxidation, human primary hepatocytes were stimulated with palmitic acids (0.4 mM) in the presence or absence of C12 1% (v/w). We observed a decreased deposition of lipid droplets in human primary hepatocytes stimulated with C12 1% (v/w) in combination with palmitic acids (0.4 mM) when compared with cells treated with palmitic acid (0.4 mM) alone (FIG. 32, Panels A and B). Concomitantly, we observed a significant reduction of three key enzymes involved in de novo lipogenesis (ACC1, DGAT and FASN) and a significant increase of the key enzyme involved in fatty acid beta-oxidation (CPT1A) in primary human hepatocytes treated with C12 1% (v/w) and palmitic acid (0.4 mM) when compared with cells treated with palimitic acid (0.4 mM) alone (FIG. 32, Panels C and D). Similar results were also observed in our in vivo experiments. Indeed, when compared to the control group, rats treated with C12 showed a decrease in key enzymes involved in de novo lipogenesis (ACC1, DGAT and FASN) and a significant increase of the key enzyme in fatty acid beta-oxidation (CPT1A) (FIG. 33, Panels A and B). Moreover, rats treated with C12 showed a decrease gene expression of citrate transporter SLC13A5 when compared with the control group (FIG. 33, Panel C). Data from in vivo study 1 and 2 were pooled together.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A hepatoprotective composition, comprising:
a nutritionally or pharmaceutically acceptable carrier in combination with a medium-chain dicarboxylic acid, wherein the composition is formulated for oral administration;
wherein a therapeutically effective unit dose of the medium-chain dicarboxylic acid provides no more than 5% of a standard daily caloric intake of a mammal.

2. (canceled)

3. The composition of claim 1, wherein the composition is formulated as a ready-to-use drink or as a solid supplement or powder.

4. The composition of claim 1, wherein the medium-chain dicarboxylic acid is dodecanoic dicarboxylic acid.

5-7. (canceled)

8. The composition of claim 1, wherein the therapeutically effective unit dose comprises equal or less than 5 g of the medium-chain dicarboxylic acid.

9-10. (canceled)

11. The composition of claim 1, wherein the composition further comprises an additional hepatoprotective agent.

12. (canceled)

13. The composition of claim 1, wherein the therapeutically effective dose reduces risk for or progression of liver inflammation, hepatic steatosis, and/or liver fibrosis in non-alcoholic steatohepatitis (NASH), or prevents or treat non-alcoholic steatohepatitis (NASH).

14-15. (canceled)

16. A method of reducing the risk for or the progression of liver inflammation, hepatic steatosis, and/or liver fibrosis in non-alcoholic steatohepatitis (NASH) in an individual, comprising:
administering a therapeutically effective dose of a medium-chain dicarboxylic acid to the individual in need thereof;
wherein the therapeutically effective dose provides no more than 5% of a standard daily caloric intake of the individual.

17. The method of claim 16, wherein the medium-chain dicarboxylic acid is dodecanoic dicarboxylic acid, and/or wherein the individual is a human.

18. (canceled)

19. The method of claim 16, wherein the therapeutically effective dose is orally administered.

20-23. (canceled)

24. The method of claim 16, wherein the composition further comprises an additional hepatoprotective agent.

25-40. (canceled)

41. A method of treating, preventing, or reducing severity of non-alcoholic steatohepatitis (NASH) in an individual, comprising:
orally administering a therapeutically effective dose of a medium-chain dicarboxylic acid to the individual in need thereof;
wherein the therapeutically effective dose prevents or reduces severity of non-alcoholic steatohepatitis (NASH) in the individual.

42. The method of claim 41, wherein the medium-chain dicarboxylic acid is dodecanoic dicarboxylic acid or a pharmaceutically or nutraceutically acceptable salt thereof, and/or wherein the individual is a human.

43-44. (canceled)

45. The method of claim 41, wherein the therapeutically effective dose provides equal or less than 5% of a standard daily caloric intake of the individual.

46. (canceled)

47. The method of claim 41, wherein the therapeutically effective dose comprises equal or less than 5 g of the medium-chain dicarboxylic acid.

48. (canceled)

49. The method of claim 41, wherein the therapeutically effective dose further reduces the risk for or the progression of liver inflammation and/or liver fibrosis.

50. The method of claim 41, wherein the therapeutically effective dose further reduces insulin resistance.

51-65. (canceled)

66. A method of reversing liver inflammation, hepatic steatosis, and/or liver fibrosis in non-alcoholic steatohepatitis (NASH) in an individual, comprising:
orally administering a therapeutically effective dose of a medium-chain dicarboxylic acid to the individual in need thereof;
wherein the therapeutically effective dose provides no more than 5% of a standard daily caloric intake of the individual.

67. The method of claim 66, wherein the medium-chain dicarboxylic acid is dodecanoic dicarboxylic acid.

68. The method of claim 66, wherein the individual is a human.

69-73. (canceled)

74. The method of claim 66, wherein the composition further comprises an additional hepatoprotective agent.

75-85. (canceled)


SEQUENCE LISTING (12 sequences)

SEQ ID NO: 1 - DNA, 20 bp, synthetic construct
  source 1..20: mol_type = other DNA
agaggcagtggtagtcgtgt

SEQ ID NO: 2 - DNA, 20 bp, synthetic construct
  source 1..20: mol_type = other DNA
tcccctttagcccttgttcc

SEQ ID NO: 3 - DNA, 21 bp, synthetic construct
  source 1..21: mol_type = other DNA
ctacagggactggtggaatgc

SEQ ID NO: 4 - DNA, 20 bp, synthetic construct
  source 1..20: mol_type = other DNA
agcaggagtaggccccatag

SEQ ID NO: 5 - DNA, 20 bp, synthetic construct
  source 1..20: mol_type = other DNA
gaatccgcacaggctaccaa

SEQ ID NO: 6 - DNA, 20 bp, synthetic construct
  source 1..20: mol_type = other DNA
ctgggcttcaccatcaccat

SEQ ID NO: 7 - DNA, 20 bp, synthetic construct
  source 1..20: mol_type = other DNA
tggggaagagacagacacca

SEQ ID NO: 8 - DNA, 20 bp, synthetic construct
  source 1..20: mol_type = other DNA
atcgtggtagagccagacct

SEQ ID NO: 9 - DNA, 20 bp, synthetic construct
  source 1..20: mol_type = other DNA
ccacaactaccatcacgcct

SEQ ID NO: 10 - DNA, 21 bp, synthetic construct
  source 1..21: mol_type = other DNA
caggaactcagaagcccagaa

SEQ ID NO: 11 - DNA, 24 bp, synthetic construct
  source 1..24: mol_type = other DNA
aggactggtctttctatctcttgt

SEQ ID NO: 12 - DNA, 21 bp, synthetic construct
  source 1..21: mol_type = other DNA
acctccatgatgctgcttaca